(19) United States
(12) Patent Application Publication
(10) Pub. No.: US 2007/0299041 A1
(43) Pub. Date: Dec. 27, 2007

(54) INDUCTION OF INNATE IMMUNITY BY VITAMIN D3 AND ITS ANALOGS

(75) Inventors: Adrian F. Gombart, Culver City, CA (US); H Phillip Koeffler, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 11/568,964

(22) PCT Filed: May 23, 2005
(86) PCT No.: PCT/US05/18172

§ 371 Date: Nov. 10, 2006

Related U.S. Application Data
(60) Provisional application No. 60/575,030, filed on May 26, 2004.

(51) Int. Cl. A61K 31/593; A61P 31/04

(52) U.S. Cl. 514/167

(57) ABSTRACT

Cationic antimicrobial peptides (AMPs) are an integral part of the innate immune system. Cathelicidin and defensin homologs from a variety of species exhibit broad-range bactericidal activity. The human cathelicidin analog, hCAP18, is encoded by the CAMP gene. Vitamin D3 and its analogs upregulate transcription of CAMP and defensin B2 (defB2) genes, leading to increased expression of hCAP18 mRNA and defB2. Induction of CAMP was observed in acute myeloid leukemia (AML), immortalized keratinocyte and colon cancer cell lines, as well as normal human bone marrow (BM)-derived macrophages and fresh BM cells. The present invention provides methods of inducing cathelicidin production by administering Vitamin D3 or Vitamin D3 analogs, as well as methods of treating skin infections and infections of the colon, sepsis and wound healing, preventing bacterial growth on skin grafts, promoting angiogenesis, and promoting chemoattraction by administering Vitamin D3 or Vitamin D3 analogs to upregulate cathelicidin and defensin expression.

### GOVERNMENT RIGHTS

The U.S. Government has certain rights in this invention pursuant to Grant No. CA26038-20 awarded by the NIH.

### FIELD OF THE INVENTION

The invention relates to the field of innate immunity; more specifically, to the use of cationic antimicrobial peptides to affect innate immunity.

### BACKGROUND

A major concern for public health in both developed and developing countries is the alarming increase of antibiotic resistance in bacteria (Hancock, R. E. et al., “Clinical development of cationic antimicrobial peptides: from natural to novel antibiotics,” Curr Drug Targets Infect Disord, Vol. 2, pp. 79-83 (2002)). Drug resistant bacteria such as Pseudomonas aeruginosa and Staphylococcus aureus pose serious problems for immunocompromised persons and are major sources of life-threatening nosocomial infections. In 2000, nearly 660,000 cases of sepsis developed in the United States. This resulted in an in-hospital mortality rate of nearly 18% (Martin, G. S. et al., “The epidemiology of sepsis in the United States from 1979 through 2000,” N Engl J Med, Vol. 348, pp. 1546-1554 (2003)). In addition, among survivors of sepsis, an increased risk of death and decreased quality of life occurred after discharge from the hospital (Quartin, A. A. et al., “Magnitude and duration of the effect of sepsis on survival. Department of Veterans Affairs Systemic Sepsis Cooperative Studies Group,” JAMA, Vol. 277, pp. 1058-1063 (1997); Perl, T. M. et al., “Long-term survival and function after suspected gram-negative sepsis,” JAMA, Vol. 274, pp. 338-345 (1995)).

This impending crisis has spurred the search for new therapeutic agents to combat antibiotic resistance. The innate immune system of mammals provides a rapid response to repel assaults from numerous infectious agents including bacteria, viruses, fungi and parasites (Boman, H. G., “Antibacterial peptides: basic facts and emerging concepts,” J Intern Med, Vol. 254, pp. 197-215 (2003)). It provides animals the capacity to repel assaults quickly from numerous infectious agents including bacteria, viruses, fungi and parasites (Zasloff, M., “Innate immunity, antimicrobial peptides, and protection of the oral cavity,” Lancet, Vol. 360, pp. 1116-1117 (2002); Lehrer, R. I. et al., “Cathelicidins: a family of endogenous antimicrobial peptides,” Curr Opin Hematol, Vol. 9, pp. 18-22 (2002); Hancock, R. E. et al., “The role of cationic antimicrobial peptides in innate host defences,” Trends Microbiol, Vol. 8, pp. 402-410 (2000); Lehrer, R. I. et al., “Antimicrobial peptides in mammalian and insect host defence,” Curr Opin Immunol, Vol. 11, pp. 23-27 (1999); Hancock, R. E. et al., “The role of antimicrobial peptides in animal defenses,” Proc Natl Acad Sci USA, Vol. 97, pp. 8856-8861 (2000); Andreu, D. et al., “Animal antimicrobial peptides: an overview,” Biopolymers, Vol. 47, pp. 415-433 (1998)). A major component of this system is a diverse combination of cationic antimicrobial peptides (AMPs) that include the α- and β-defensins and cathelicidins. Because bacteria have difficulty developing resistance against AMPs and are quickly killed by them, this class of antimicrobial agents is being commercially developed as a source of peptide antibiotics (Hancock, R. E. (2002); Hancock, R. E. et al., “Cationic peptides: a new source of antibiotics,” Trends Biotechnol, Vol. 16, pp. 82-88 (1998); Zasloff, M., “Antimicrobial peptides in health and disease,” N Engl J Med, Vol. 347, pp. 1199-1200 (2002)). The majority of the pharmaceutical effort has concentrated on the development of topically applied agents (Zasloff, M. (2002)). However, the expense and difficulty of preparing large amounts of peptide and the uncertainty in systemic use of these peptides has slowed their development beyond topical treatments.

Mammals express two broad classes of peptide antibiotics, cathelicidins and defensins (Nagaoka 2002). These peptide antibiotics exhibit potent antimicrobial effects against gram-positive and gram-negative bacteria, fungi, and viruses (Hancock 2000b). Many human and mouse β-defensin genes have been reported, and the existence of additional β-defensin genes is suspected because of the high frequency of gene duplication within β-defensin clusters (Schutte, B. C. et al., “Discovery of five conserved β-defensin gene clusters using a computational search strategy,” Proc Natl Acad Sci USA, Vol. 99, pp. 2129-2133 (2002)). Cathelicidin homologs have been identified in a variety of species, including rabbits (CAP18) (Larrick, J. W. et al., “Complementary DNA sequence of rabbit CAP18—a unique lipopolysaccharide binding protein,” Biochem Biophys Res Commun, Vol. 179, pp. 170-175 (1991)), mice (mCRAMP) (Gallo, R. L. et al., “Identification of CRAMP, a cathelin-related antimicrobial peptide expressed in the embryonic and adult mouse,” J Biol Chem, Vol. 272, pp. 13088-13093 (1997); Popsueva, A. E. et al., “A novel murine cathelin-like protein expressed in bone marrow,” FEBS Lett, Vol. 391, pp. 5-8 (1996)), rats (rCRAMP), sheep (SMAP29 and SMAP34) (Bagella, L. et al., “cDNA sequences of three sheep myeloid cathelicidins,” FEBS Lett, Vol. 376, pp. 225-228 (1995); Huttner, K. M., et al. 1998. Localization and genomic organization of sheep antimicrobial peptide genes. Gene 206:85-91; Mahoney, M. M., Lee, A. Y., Brezinski-Caliguri, D. J., Huttner, K. M. 1995. Molecular analysis of the sheep cathelin family reveals a novel antimicrobial peptide. FEBS Lett 377:519-522; Skerlavaj, B., et al. 1999. SMAP-29: a potent antibacterial and antifungal peptide from sheep leukocytes. FEBS Lett 463:58-62), pigs (PMAP-36 and PMAP-37) (Storici, P., et al. 1994. Chemical synthesis and biological activity of a novel antibacterial peptide derived from pig myeloid cDNA. FEBS Lett 337:303-307; Tossi, A., et al. 1995. PMAP-37, a novel antibacterial peptide from pig myeloid cells. cDNA cloning, chemical synthesis and activity. Eur J Biochem 228:941-946), cows (BMAP-27 and BMAP-28) (Skerlavaj, B., et al. 1996. Biological characterization of two novel cathelicidin-derived peptides and identification of structural requirements for their antimicrobial activity and cell lytic activity. J Biol Chem 271:28375-28381), and humans (hCAP18) (Agerberth, B., et al. 1995. FALL-39, a putative human peptide antibiotic, is cysteine-free and expressed in bone marrow and testis. Proc Natl Acad Sci USA 92:195-199; Larrick, J. W., et al. 1995. Human CAP18: a novel antimicrobial lipopolysaccharide-binding protein. Infect Immun 63:1291-1297). Each of these peptides exhibits broad-spectrum bactericidal activity that appears to be mediated by disruption of the bacterial membrane (Oren, Z., Shai, Y. 1998. Mode of action of linear amphipathic α-helical antimicrobial peptides. Biopolymers 47:451-463). Cathelicidins are produced as precursors (propeptides) that require proteolytic processing to generate a mature antimicrobial peptide (Travis, S. M., et al. 2000. Bactericidal activity of mammalian cathelicidin-derived peptides. Infect Immun 68:2748-2755). Although these peptides lack the ability to recognize specific antigens, their fast delivery to the site of infections, wounds, and inflammation makes them an integral part of innate immunity (Boman, H. G. 1995. Peptide antibiotics and their role in innate immunity. Ann Rev Immunol 13:61-92).

One class of β-defensin genes that show promise is known as defensin β2 genes (defB2) (Wang, T. T. et al., “Cutting Edge: 1,25-Dihydroxyvitamin D3 is a Direct Inducer of Antimicrobial Peptide Gene Expression,” J Immunol (2004)). The β-defensins are defined by a six-cysteine motif and a large number of basic amino acid residues. Their coding sequences consist of two exons. The first exon includes the 5′ untranslated region and encodes the leader domain of the preproprotein; the second exon encodes the mature peptide with the six-cysteine domain (Schutte, B. C. et al., “Discovery of five conserved β-defensin gene clusters using a computational search strategy,” Proc Natl Acad Sci USA, Vol. 99, pp. 2129-2133 (2002)). One AMP that shows promise is the human cathelicidin antimicrobial peptide (CAMP) also known as hCAP18/LL-37/FALL-39. It is the only known human cathelicidin. The C-terminal domain of cathelicidin peptides comprises an antimicrobial peptide (AMP) domain, while the N-terminal comprises the highly conserved cathelin domain (Zanetti, M., Gennaro, R., Romeo, D. 1995. Cathelicidins: a novel protein family with a common proregion and a variable C-terminal antimicrobial domain. FEBS Lett 374:1-5; Zanetti, M., Gennaro, R., Romeo, D. 1997. The cathelicidin family of antimicrobial peptide precursors: a component of the oxygen-independent defense mechanisms of neutrophils. Ann NY Acad Sci 832:147-162). hCAP18 has been isolated from specific granules of human neutrophil granulocytes (Cowland, J. B., Johnson, A. H., Borregaard, N. 1995. hCAP-18, a cathelin/bactenecin like protein of human neutrophil specific granules. FEBS Lett 368:173-176; Gudmundsson, G. H., et al. 1996. The human gene FALL39 and processing of the cathelin precursor to the antibacterial peptide LL-37 in granulocytes. Eur J Biochem 238:325-332). The cathelicidins are a family of proteins consisting of a C-terminal cationic AMP domain that is activated by cleavage from the N-terminal cathelin portion of the propeptide. The C-terminal antimicrobial peptide in the human cathelicidin hCAP18 (human cationic antibacterial protein of 18 kDa) is the 37 amino acid residue peptide LL-37 (Zanetti, M., Gennaro, R., Romeo, D. 1995. Cathelicidins: a novel protein family with a common proregion and a variable C-terminal antimicrobial domain. FEBS Lett 374:1-5; Gudmundsson, G. H., Agerberth, B. 1999. Neotrophil antibacterial peptides, multifunctional effector molecules in the mammalian immune system. J Immunol Methods 232:45-54; Gennaro, R., Zanetti, M. 2000. Structural features and biological activities of the cathelicidin-derived antimicrobial peptides. Biopolymers 55:31-49; Lehrer, R. I., Ganz, T. 2002. Cathelicidins: a family of endogenous antimicrobial peptides. Curr Opin Hematol 9:18-22), which is generated by proteinase-3 cleavage of hCAP18 (Sorensen, O. et al. 2001. Human cathelicidin, hCAP-18, is processed to the antimicrobial peptide LL-37 by extracellular cleavage with proteinase 3. Blood 97:3951-3959). The majority of the CAMP propeptide is stored in secondary or specific granules of neutrophils from which it can be released at sites of microbial infection (Sorensen, O. et al., “The human antibacterial cathelicidin, hCAP-18, is synthesized in myelocytes and metamyelocytes and localized to specific granules in neutrophils,” Blood, Vol. 90, pp. 2796-2803 (1997)). In addition to neutrophils, various white blood cell populations express hCAP18. These include natural killer cells, γδT cells, B-cells, monocytes (Agerberth, B. et al., “The human antimicrobial and chemotactic peptides LL-37 and alpha-defensins are expressed by specific lymphocyte and monocyte populations,” Blood, Vol. 96, pp. 3086-3093 (2000)) and mast cells (Di Nardo, A. et al., “Cutting edge: mast cell antimicrobial activity is mediated by expression of cathelicidin antimicrobial peptide,” J Immunol, Vol. 170, pp. 2274-2278 (2003)). CAMP/hCAP18 is secreted into the blood and significant levels are found in the plasma (Sorensen, O. et al., “An ELISA for hCAP-18, the cathelicidin present in human neutrophils and plasma,” J Immunol Methods, Vol. 206, pp. 53-59 (1997)).

Also, CAMP is synthesized and secreted in significant amounts by those tissues that are exposed to environmental microbes. This includes the squamous epithelia of the mouth, tongue, esophagus, lungs, intestine, cervix and vagina (Frohm, N. M. et al., “The human cationic antimicrobial protein (hCAP18), a peptide antibiotic, is widely expressed in human squamous epithelia and colocalizes with interleukin-6,” Infect Immun, Vol. 67, pp. 2561-2566 (1999); Bals, R. et al., “The peptide antibiotic LL-37/hCAP-18 is expressed in epithelia of the human lung where it has broad antimicrobial activity at the airway surface,” Proc Natl Acad Sci USA, Vol. 95, pp. 9541-9546 (1998)). In addition, it is produced by salivary and sweat glands (Murakami, M. et al., “Cathelicidin antimicrobial peptides are expressed in salivary glands and saliva,” J Dent Res, Vol. 81, pp. 845-850 (2002)), epididymis, testis (Maim, J. et al., “The human cationic antimicrobial protein (hCAP-18) is expressed in the epithelium of human epididymis, is present in seminal plasma at high concentrations, and is attached to spermatozoa,” Infect Immun, Vol. 68, pp. 4297-4302 (2000)) and mammary glands (Murakami, M. et al., “Expression and secretion of cathelicidin antimicrobial peptides in murine mammary glands and human milk,” Pediatr Res, Vol. 57, pp. 10-15 (2005); Armogida, S. A. et al., “Identification and quantification of innate immune system mediators in human breast milk,” Allergy Asthma Proc, Vol. 25, pp. 297-304 (2004); Hammami-Hamza, S. et al., “Cloning and sequencing of SOB3, a human gene coding for a sperm protein homologous to an antimicrobial protein and potentially involved in zona pellucida binding,” Mol Hum Reprod, Vol. 7, pp. 625-632 (2001)). Expression in these tissues results in secretion of the polypeptide in wounds (Frohm, M. et al., “Biochemical and antibacterial analysis of human wound and blister fluid,” Eur J Biochem, Vol. 237, pp. 86-92 (1996)), sweat (Murakami, M. et al., “Cathelicidin anti-microbial peptide expression in sweat, an innate defense system for the skin,” J Invest Dermatol, Vol. 119, pp. 1090-1095 (2002)), airway surface fluids (Bals, R. (1998)), seminal plasma (Andersson, E. et al., “Isolation of human cationic antimicrobial protein-18 from seminal plasma and its association with prostasomes,” Hum Reprod, Vol. 17, pp. 2529-2534 (2002)) and milk (Murakami, M. (2005); Armogida, S. A. (2004)). CAMP/hCAP18 possesses several important activities including bactericidal, anti-sepsis, chemoattraction, and promotion of angiogenesis and wound healing. Thus, the possibility of extrinsically manipulating endogenous expression of CAMP for systemic and localized therapeutic benefit is very attractive.

Since their discovery more than a decade ago, the majority of expression studies have been focused on the detection of cathelicidins in various tissues; however, the transcriptional mechanisms that regulate cathelicidin gene expression have not been adequately elucidated. Understanding the signaling pathways and the downstream transcription factors that regulate CAMP gene expression in a tissue-specific manner is crucial for designing approaches for therapeutic manipulation of endogenous gene expression. Because AMPs serve a role in host defense and may act as mediators of other biological processes, their expression is tightly regulated.

A number of studies indicate that CAMP and hCAP18 play an important role in defending against infection. Expression of the CAMP gene is upregulated during cutaneous injection, injury, or inflammation (psoriasis) (Dorschner, R. A. et al., “Cutaneous injury induces the release of cathelicidin anti-microbial peptides active against group A Streptococcus,” J Invest Dermatol, Vol. 117, pp. 91-97 (2001); Ong, P. Y. et al., “Endogenous antimicrobial peptides and skin infections in atopic dermatitis,” N Engl J Med, Vol. 347, pp.1151-1160 (2002); Frohm, M. et al., “The expression of the gene coding for the antibacterial peptide LL-37 is induced in human keratinocytes during inflammatory disorders,” J Biol Chem, Vol. 272, pp. 15258-15263 (1997)). Decreased levels of hCAP18 in the skin of individuals with atopic dermatitis correlates with increased susceptibility to skin infection compared to individuals with psoriasis (Ong, P. Y. (2002)). Vitamin D3 and its analogs have proven safe and effective in the treatment of psoriasis. Treatment of CAMP-deficient atopic dermatitis with vitamin D₃ may prove beneficial, also. Mice deficient in the murine homolog CRAMP are much more susceptible to skin infection than wild type mice (Nizet, V. et al., “Innate antimicrobial peptide protects the skin from invasive bacterial infection,” Nature, Vol. 414, pp. 454-457 (2001)). Chronic oral bacterial infections occur in morbus Kostmann patients who suffer from a severe chronic neutropenia. Neutrophils from these patients lack CAMP expression (Putsep, K., Carlsson, G., Boman, H. G., Andersson, M. 2002. Deficiency of antibacterial peptides in patients with morbus Kostmann: an observation study. Lancet 360:1116-1117). Patients suffering from specific granule deficiency (SGD) lack expression of both hCAP18 and defensins, and they suffer severe, recurrent bacterial infections (Gombart, A. F., Koeffler, H. P. 2002. Neutrophil specific granule deficiency and mutations in the gene encoding transcription factor C/EBP(epsilon). Curr Opin Hematol 9:36-42). An increase in the expression of LL-37 and other antimicrobial peptides in cultured composite keratinocyte skin grafts enhances the ability of the keratinocytes to combat infection in a burn wound site (Erdag, G., Morgan, J. R. 2002. Interleukin-1α and interleukin-6 enhance the antibacterial properties of cultured composite keratinocyte grafts. Ann Surg 235:113-124). Protective effects of CAMP overexpression in respiratory epithelia were observed in a cystic fibrosis model (Bals, R. et al., “Transfer of a cathelicidin peptide antibiotic gene restores bacterial killing in a cystic fibrosis xenograft model,” J Clin Invest, Vol. 103, pp. 1113-1117 (1999)). The systemic expression of CAMP/hCAP18 in mice improved survival rates following intravenous injection of lipopolysaccharide (LPS) (Bals, R. et al., “Augmentation of innate host defense by expression of a cathelicidin antimicrobial peptide,” Infect Immun, Vol. 67, pp. 6084-6089 (1999)). LPS is a component of the bacterial cell wall of gram-negative bacteria such as E. coli or P. aeruginosa. Massive gram-negative bacterial infection can result in septic shock due to the large amounts of LPS present in the blood. Thus, hCAP18 may not only aid in clearance of bacterial infection, but may protect against the sepsis. This protection probably derives from the ability of CAMP to bind to LPS and neutralize it (Larrick, J. W. et al., “Human CAP18: a novel antimicrobial lipopolysaccharide-binding protein,” Infect Immun, Vol. 63, pp. 1291-1297 (1995); Kirikae, T. et al., “Protective effects of a human 18-kilodalton cationic antimicrobial protein (CAP18)-derived peptide against murine endotoxemia,” Infect Immun, Vol. 66, pp. 1861-1868 (1998); Turner, J. et al., “Activities of LL-37, a cathelin-associated antimicrobial peptide of human neutrophils,” Antimicrob Agents Chemother, Vol. 42, pp. 2206-2214 (1998); Scott, M.G. et al., “The human antimicrobial peptide LL-37 is a multifunctional modulator of innate immune responses,” J Immunol, Vol. 169, pp. 3883-3891 (2002)). The hCAP18 peptide has been shown to inhibit LPS-induced cellular responses such as release of TNF-α, tissue factor and nitric oxide, thus protecting mice and pigs from septic shock (Larrick, J. W. (1995); VanderMeer, T. J. et al., “Protective effects of a novel 32-amino acid C-terminal fragment of CAP18 in endotoxemic pigs,” Surgery, Vol. 117, pp. 656-662 (1995)). In vitro, hCAP18 inhibits macrophage activation by LPS and other bacterial components (Scott, M. G. (2002)).

In addition to its antimicrobial and LPS binding activities, hCAP18 is increasingly associated with a wide range of biological effects (FIG. 15). The discovery of additional activities for hCAP18 indicates that it may have a broader role in host defense than previously suspected. In vitro studies have shown that the LL-37 domain of hCAP18 induces migration of human peripheral blood monocytes, neutrophils, CD4 T cells, and rat mast cells (Agerberth, B., et al. 2000. The human antimicrobial and chemotactic peptides LL-37 and α-defensins are expressed by specific lymphocyte and monocyte populations. Blood 96:3086-3093; De Yang 2000. LL-37, the neutrophil granule- and epithelial cell-derived cathelicidin, utilizes formyl peptide receptor-like 1 (FPRL1) as a receptor to chemoattract human peripheral blood neutrophils, monocytes, and T cells. J Exp Med 192:1069-1074; Niyonsaba, F., et al. 2002. A cathelicidin family of human antibacterial peptide LL-37 induces mast cell chemotaxis. Immunology 106:20-26). In addition, it stimulates histamine release and intracellular Ca²⁺ mobilization in rat mast cells (Niyonsaba (2002)). LL-37 has also been shown to alter transcription of both pro- and anti-inflammatory genes in murine macrophage and human epithelial cell lines (Scott, M. G., et al. 2002. The human antimicrobial peptide LL-37 is a multifunctional modulator of innate immune responses. J Immunol 169:3883-3891), and to promote wound neovascularization (pro-angiogenic properties) and re-epithelialization of healing skin (Heilborn, J. D., et al. 2003. The cathelicidin anti-microbial peptide LL-37 is involved in re-epithelialization of human skin wounds and is lacking in chronic ulcer epithelium. J Invest Dermatol 120:379-389; Koczulla, R., et al. 2003. An angiogenic role for the human peptide antibiotic LL-37/hCAP18. J Clin Invest 111:1665-1672).

Vitamin D is the generic term for a family of secosteroid hormones that exhibit affinity for the nuclear Vitamin D receptor (VDR). VDR is a member of the steroid/thyroid hormone superfamily, and contains a highly conserved N-terminal DNA binding domain and a less conserved C-terminal ligand binding domain. VDR is a ligand-activated transcription factor that binds to a Vitamin D response element (VDRE) in the promoter or enhancer region of target genes. The VDRE consensus sequence consists of two six nucleotide half sites separated by three nucleotides (Jehan, F., DeLuca, H. F. 1997. Cloning and characterization of the mouse vitamin D receptor promoter. Proc Natl Acad Sci USA 94:10138-10143).

The members of the Vitamin D family function to regulate calcium and phosphate metabolism, mediating their effect in large part by stimulating intestinal calcium absorption. One member of the Vitamin D family, Vitamin D₃ [1α,25(OH)₂D₃], has been shown to stimulate cell differentiation and inhibit excessive cell proliferation in a variety of cells (Abe, E., et al. 1981. Differentiation of mouse myeloid leukemia cells induced by 1 alpha,25-dihydroxyvitamin D3. Proc Natl Acad Sci USA 78:4990-4994). The central role of Vitamin D₃ in calcium metabolism, cell proliferation, and cell differentiation has made it an attractive candidate for the treatment of a variety of diseases, including cancer, osteoporosis, hyperparathyroidism, and psoriasis. Unfortunately, high levels of Vitamin D₃ are toxic because they cause overabsorption of calcium, a condition known as hypercalcemia (Norman, A. W. 1995. The vitamin D endocrine system: manipulation of structure-function relationships to provide opportunities for development of new cancer chemopreventive and immunosuppressive agents. J Cell Biochem Suppl 22:218-225). This has led to the development of a wide variety of Vitamin D₃ analogs (deltanoids) for the treatment of various disorders (Posner, G. H., “Low-Calcemic Vitamin D Analogs (Deltanoids) for Human Cancer Prevention,” J. Nutr., Vol. 132, pp. 3802S-3803S (2002)). Several of these analogs have been approved for use in patients, including calcipotriol for the treatment of psoriasis (U.S. Pat. No. 5,292,727), calcitol and paracalcitol for the treatment of hyperthyroidism (U.S. Pat. Nos. 4,308,264 and 5,246,925, respectively), doxercalciferol for reduction of elevated parathyroid hormone levels (U.S. Pat. No. 4,555,364), 22-oxacalcitrol, and alfacalcidol (Brown, A. J. 2001. Therapeutic uses of Vitamin D analogues. Am J Kidney Dis 38(5Suppl5):S3-S19).

The foregoing examples of the related art and limitations related therewith are intended to be illustrative and not exclusive. Other limitations of the related art will become apparent to those of skill in the art upon a reading of the specification and a study of the drawings.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

hCAP18, a member of the cathelicidin family of peptides, is known to possess antimicrobial and antiseptic properties, as well as the ability to promote wound healing, angiogenesis, and chemoattraction. In various embodiments, methods of increasing endogenous levels of cathelicidins such as hCAP18 by administering Vitamin D₃ and/or Vitamin D₃ analogs are disclosed. In other embodiments, methods of increasing endogenous levels of defensins such as defensin β2 (defB2) by administering Vitamin D₃ and/or Vitamin D₃ analogs are disclosed.

An embodiment by way of non-limiting example includes a method of inducing endogenous cellular cathelicidin and/or defensin production by administering Vitamin D₃. In various embodiments, induction of cathelicidin and/or defensin production occurs at the transcriptional level. In various embodiments, the cathelicidin being induced is hCAP18. In various embodiments, the defensin being induced is defB2.

Another embodiment by way of non-limiting example includes a method of inducing endogenous cellular cathelicidin and/or defensin production by administering one or more Vitamin D₃ analogs or a combination of Vitamin D₃ and one or more Vitamin D₃ analogs. In various embodiments, induction of cathelicidin and/or defensin production occurs at the transcriptional level. In various embodiments, the cathelicidin being induced is hCAP18. In various embodiments, the defensin being induced is defB2. In various embodiments, the Vitamin D₃ analog(s) being administered is chosen from the group consisting of lexacalcitol (KH1060), seocalcitol (EB1089), and Vitamin D₃ analog I (1,25R,26-(OH)₂-22-ene-D₃).

Another embodiment by way of non-limiting example includes a method of inducing endogenous cathelicidin and/or defensin production in a subject by administering Vitamin D₃. In various embodiments, induction of cathelicidin and/or defensin production occurs at the transcriptional level. In various embodiments, the cathelicidin being induced is hCAP18. In various embodiments, the defensin being induced is defB2. In various embodiments, induction of cathelicidin and/or defensin production treats skin infections and infections of the colon, sepsis and wound healing, prevents bacterial growth on skin grafts, promotes angiogenesis, and promotes chemoattraction. In various embodiments, the subject is human and the route of administration is topical, transdermal, or parenteral. In various embodiments, the subject is a mammal or primate and the route of administration is topical, transdermal, or parenteral.

Another embodiment by way of non-limiting example includes a method of inducing endogenous cathelicidin and/or defensin production in a subject by administering one or more Vitamin D₃ analogs or a combination of Vitamin D₃ and one or more Vitamin D₃ analogs. In various embodiments, induction of cathelicidin and/or defensin production occurs at the transcriptional level. In various embodiments, the cathelicidin being induced is hCAP18. In various embodiments, the defensin being induced is defB2. In various embodiments, induction of cathelicidin and/or defensin production treats skin infections and infections of the colon, sepsis and wound healing, prevents bacterial growth on skin grafts, promotes angiogenesis, and promotes chemoattraction. In various embodiments, the subject is human and the route of administration is topical, transdermal, or parenteral. In various embodiments, the subject is a mammal or primate and the route of administration is topical, transdermal, or parenteral. In various embodiments, the Vitamin D₃ analog(s) being administered is chosen from the group consisting of lexacalcitol (KH1060), seocalcitol (EB1089), and Vitamin D₃ analog I.

Another embodiment by way of non-limiting example includes a method of treating skin infections and infections of the colon, sepsis, wounds or bacterial growth on skin grafts by administering Vitamin D₃, one or more Vitamin D₃ analogs, or a combination thereof to a subject and inducing transcription of cathelicidin and/or defensin. In various embodiments, the subject is human and the cathelicidin being induced is hCAP18. In various embodiments, the subject is human and the defensin being induced is defB2. In various embodiments, the subject is a mammal or primate. In various embodiments, administration occurs at the site of sepsis, microbial infection, or wound, preferably in the neutrophils, plasma, epithelial cells, or oral cavity of the subject. In various embodiments, administration occurs at a site other than the site of sepsis, microbial infection, or wound. In various embodiments, Vitamin D₃ and/or Vitamin D₃ analogs reach the site of the sepsis, microbial infection, or wound by traveling through the circulatory system. In various embodiments, administration is topical, transdermal, or parenteral. In various embodiments, administration occurs in an effective amount until the condition is treated, and Vitamin D₃ and/or Vitamin D₃ analogs are administered in a pharmaceutically acceptable carrier.

In addition to the exemplary aspects and embodiments described above, further aspects and embodiments will become apparent by reference to the drawings and by study of the following detailed descriptions.

### BRIEF DESCRIPTION OF DRAWINGS

Exemplary embodiments are illustrated in referenced figures of the drawings. It is intended that the embodiments and figures disclosed herein are to be considered illustrative rather than restrictive.

FIG. 1: Induction of CAMP mRNA expression by 1,25[OH]₂D₃. U937 cells were treated either with vehicle (−, ethanol) or the indicated compounds for 24 h as described in the EXAMPLES. Expression levels of CAMP were determined by QRT-PCR. Standard curves with known amounts of CAMP or 18S cDNA were included to measure the starting quantity of CAMP (ng) and 18S (ng) cDNA in each sample. The graphs depict the ratio of CAMP/18S in each sample (+/−SD). PCR was performed in triplicate for each sample.

FIG. 2: Vitamin D₃-mediated induction of CAMP mRNA expression in acute myeloid leukemia cell lines. Acute myeloid leukemia (AML) cell lines HL60 and U937 were treated with 1×10⁻⁷ M Vitamin D₃ (“D3”) for various time periods. Total RNA at each time point was isolated and electrophoresed, then transferred to a charged membrane for Northern analysis. Blots were sequentially probed with ³²P-labeled DNA probes specific for CAMP, CDIIb, and β-actin mRNAs. The β-actin probe (lower panel in A and B) served as a control to demonstrate that each lane contained similar levels of RNA. The CDIIb probe (middle panel in A and B) served to confirm that Vitamin D₃ induced monocytic differentiation as expected. In FIG. 2A, CAMP mRNA levels were measured at 0, 1, 3, and 5 days after Vitamin D₃ addition. CAMP mRNA expression was observed in both cell lines on day 1, but this expression was substantially higher in the U937 cell line. In FIG. 2B, CAMP mRNA levels in the U937 cell line were measured at 0, 1, 3, 6, 12, and 24 hours after Vitamin D₃ addition. CAMP mRNA expression was observed between 1 and 3 hours.

FIG. 3: Dose responsiveness of Vitamin D₃-mediated induction of CAMP mRNA expression. AML cell lines U937, HL60, and NB4 were treated with dosages of Vitamin D₃ (“D3”) ranging from 1×10⁻⁶ M to 1×10⁻⁹ M. 24 hours after treatment, RNA was isolated, electrophoresed, and analyzed by Northern blot. Blots were sequentially probed with ³²P-labeled DNA probes specific for CAMP, CDIIb, and β-actin mRNAs. A strong, Vitamin D₃ dose-dependent induction of CAMP mRNA expression was observed in U937 cells. Moderate induction was observed in HL60 and NB4 cells.

FIG. 4: Vitamin D₃ analog-mediated induction of CAMP mRNA expression in AML cell lines. U937 cells were treated with 1×10⁻⁷ M Vitamin D₃ or Vitamin D₃ analog (KH1060, EB 1089, or I) for various time periods. Total RNA was isolated at 12 and 24 hours, electrophoresed, and analyzed by Northern blot. Blots were sequentially probed with ³²P-labeled DNA probes specific for CAMP or β-actin mRNAs. Induction of CAMP mRNA expression was observed for each of the three Vitamin D₃ analogs tested.

FIG. 5: Induction of CAMP mRNA expression by 1,25[OH]₂D₃. U937 cells were treated either with vehicle (0) or 1×10⁻⁷ M 1,25[OH]₂D₃ for 1, 2, 4 or 6 h in the absence (−ActD) or presence (+ActD) of actinomycin D (10 μg/ml). Expression levels of CAMP were determined by QRT-PCR and normalized to 18S.

FIG. 6: Vitamin D₃-mediated induction of CAMP mRNA expression in the absence of protein synthesis. U937 cells were treated with 1×10⁻⁷ M Vitamin D₃ for varying time periods in the absence (−) or presence (+) of 20 μg/ml cyclohexamide (CHX). Total RNA from 0, 6, and 9 hours was isolated, electrophoresed, and analyzed by Northern blot. Blots were sequentially probed with ³²P-labeled DNA probes specific for CAMP and β-actin mRNAs. Vitamin D₃-mediated induction of CAMP mRNA expression was not impaired by the presence of CHX.

FIG. 7: Induction of CAMP mRNA expression by 1,25[OH]₂D₃. U937 cells were treated either with vehicle (0) or 1×10⁻⁷ M 1,25[OH]₂D₃ for 12 or 24 h. The cDNAs from total RNA were analyzed by RT-PCR using primers against myeloperoxidase (MPO), α-defensin (HNP-3), matrix metalloprotease 8 (MMP8), lactoferrin (LTF), CAMP, β-actin and 18S. Amplification for all genes was 35 cycles except CAMP (30 cycles), β-actin (25 cycles) and 18S (10 cycles). A negative control (c, ddH₂O) and a positive control (normal bone marrow RNA, BM) were included.

FIG. 8: CAMP mRNA induction occurs in the presence of Vitamin D₃. The myeloid leukemia cell lines HL60 and U937 were treated either with vehicle (0), 1,25[OH]₂D₃ (1×10⁻⁷ M) or TPA (5 ng/ml) for 1, 3 or 5 days. Total RNA was extracted and analyzed by Northern blot. Blots were sequentially probed with ³²P-labeled DNA probes specific for CAMP, CDIIb, and β-actin mRNAs. CAMP mRNA expression was observed only in the presence of Vitamin D₃, not TPA

FIG. 9: Vitamin D₃-mediated induction of CAMP mRNA expression in the absence of monocytic differentiation. U937 and HL60 sublines HL60R (pan-resistant) and HL60Δ404 (ATRA-resistant) were treated either with vehicle (−) or 1,25[OH]₂D₃ (+, 1×10⁻⁷ M). Total RNA from 24 hours was isolated, electrophoresed, and analyzed by Northern blot. Blots were sequentially probed with ³²P-labeled DNA probes specific for CAMP, CDIIb, and β-actin mRNAs. Addition of Vitamin D₃ induced expression of CAMP mRNA in all three cell types.

FIG. 10: Vitamin D₃-mediated induction of CAMP mRNA expression in normal and leukemic bone marrow cells. In FIG. 10A, bone marrow cells from normal human patients (NHBM) were cultured in RPMI1640+10% FCS either with vehicle (0) or 1,25[OH]₂D₃ for either 72 or 120 h. BM-derived Mφ were treated for 24 h either with vehicle (0) or an increasing concentration of 1,25[OH]₂D₃. As a positive control for induction, U937 cells were either treated with vehicle or 1,25[OH]₂D₃ for 12 and 24 h with either vehicle (0). Total RNA was isolated from each cell type and cDNAs were synthesized by reverse transcription. These cDNAs were analyzed by RT-PCR using fluorescent probes against either CAMP or 18S. A standard curve was generated using known amounts of CAMP and 18S to determine the amount of CAMP and 18S in each sample. Vitamin D₃ induced CAMP mRNA expression in a dose-dependent manner in every cell type tested. The X-axis represents (CAMP (ng)/18S(ng)), +/−SD. The fold-change for each sample is indicated by the number within the bar. In FIG. 10B, The AML BM cells were treated for 24 h either with vehicle (0) or 1,25[OH]₂D₃. Total RNA from 0 and 24 hours was isolated and cDNA was synthesized by reverse transcription. This cDNA was analyzed by RT-PCR using fluorescent probes against either CAMP or 18S. A standard curve was generated using known amounts of CAMP and 18S to determine the amount of CAMP and 18S in each sample. Vitamin D₃ induced CAMP mRNA expression in a dose-dependent manner. The X-axis represents (CAMP(ng)/18S(ng)), +/−SD. The fold-change for each sample is indicated by the number within the bar.

FIG. 11: Vitamin D₃-mediated induction of CAMP mRNA expression in keratinocyte (HaCat) and colon cancer (Ht-29) cell lines. Keratinocyte (HaCat, FIG. 11A) and colon cancer (Ht-29, FIG. 11B) cells were treated either with vehicle (−) or 1,25[OH]₂D₃ (+, 1×10⁻⁷ M). Total RNA from 0 and 24 hours was isolated and cDNA was synthesized by reverse transcription. This cDNA was analyzed by QRT-PCR using fluorescent probes against either CAMP or 18S. The X-axis represents (CAMP(ng)/18S(ng)), +/− SD. The fold-change for each sample is indicated by the number within the bar.

FIG. 12: Induction of CAMP protein hCAP18 in U937 treated with vitamin D3. Cells were either treated with vehicle (−) or 1,25[OH]₂D₃ (+, 1×10⁻⁷ M) for 18 and 36 h. Cytospins of cells treated for 36 h were prepared and immunofluorescence for hCAP18 was performed. Photographs (FIG. 12A) were taken at 200× magnification. Examples of strongly positive cells are indicated by the white arrows. Total cell lysates were analyzed by Western blot (FIG. 12B) for hCAP18 expression. The position of hCAP18 is indicated by the arrow at the right of FIG. 12B, and the molecular weight markers are indicated at the left. Subsequent probing of the same blot for GAPDH demonstrated equivalent loading of protein in each lane (lower panel). FIG. 12C shows the levels of hCAP18 in the medium of U937 cells treated either with vehicle or 1,25[OH]₂D₃ determined by ELISA.

FIG. 13: Identification of a functional VDRE in the human CAMP promoter. FIG. 13A shows the sequence (SEQ ID NO.: 1) (SmaI restriction enzyme site, nucleotides 74-79; VDRE sequence 78-92; HindIII restriction enzyme site 197-202; Binding site for STAT3 254-260; Binding site for CDP 305-313; Binding site for C/EBP 490-497; Binding site for PU.1 498-507; Binding site for C/EBP 553-561; Binding site for C/EBP 645-652) of the human CAMP promoter (−693 to +14) (Larrick, J. W. et al., “Structural, functional analysis and localization of the human CAP18 gene,” FEBS Lett, Vol. 398, pp. 74-80 (1996)) as it was cloned into the firefly luciferase reporter vector pXP2. The restriction enzyme sites are indicated across the top of the sequence and the transcription factor binding sites are indicated across the top and bottom. These include CCMTT displacement protein (CDP), STAT3, C/EBP, PU.1 and VDR. The sequence of the primers used for chromatin IP are underlined (line with closed circles at each end). Two additional constructs were generated by deleting from the 5′-end with SmaI and with HindIII. The shaded box of the schematic diagram (FIG. 13B) indicates the position of a repetitive element (SINE) in the promoter. In FIG. 13C, U937 cells were transfected (two times in duplicate) with the CAMP promoter-firefly luciferase reporter constructs and a renilla expression vector, phTKRL. Each transfection was treated either with vehicle (−) or 1,25[OH]₂D₃ (+, 1×10⁻⁷ M) for 18 h. Dual luciferase assays were performed and firefly luciferase activity was normalized to renilla luciferase activity. The untreated and treated conditions for each construct were compared. Abbreviations: CAMP-Luc (pXP2-CAMP-Luc); ΔSmaI [pXP2-CAMP(ΔSmaI)-Luc] and ΔHindIII [pXP2-CAMP(ΔHindIII)-Luc]. FIGS. 13D and 13E show VDR and C/EBPε binding to the human CAMP promoter. Approximately 1×10⁷ cells were incubated in the absence (−) or presence (+) of 1,25[OH]₂D₃ at 1×10⁻⁷ M for 4 hours. ChIP assays were performed; protein/DNA complexes were cross-linked in formaldehyde for 10 minutes, and the cross-linking reaction was terminated by addition of glycine. The cells were washed in ice-cold PBS containing PMSF, re-suspended in SDS-lysis buffer containing protease inhibitors, and incubated on ice for 10 minutes. The lysates were sonicated and then pelleted at 13K RPM for 10 minutes at 4° C. 200 μl of supernatant was mixed with 1.8 ml of dilution buffer and precleared with protein A-agarose. Anti-VDR antibody, anti-C/EBPε, or preimmune serum was added and the sample was incubated overnight at 4° C. The agarose/antibody/protein/DNA complex was pelleted and washed in low salt, high salt, LiCL, and TE. The complex was removed from the protein A-agarose in elution buffer, and cross-linking was reversed in 100 mM NaCl at 65° C. for 4 hours. DNA was isolated by phenol/chloroform extraction and ethanol precipitation, and the CAMP promoter fragment was detected both by conventional (FIG. 13D) PCR (reverse image of ethidium bromide stained gel; 30 cycles) and QRT-PCR (FIG. 13E). The relative amount of CAMP promoter gDNA was determined in each sample by SYBR Green QRT-PCR. The differences (fold-change) were normalized to the preimmune (Pre, average of both untreated and treated) and indicated by the number within each bar. The positions of the DNA markers are indicated in base pairs (bp) at the left of the panel and the size of the expected promoter product is indicated at the right of the panel. Abbreviations: Anti-ε (rabbit anti-C/EBPε antiserum).

FIG. 14: Vitamin D₃ induction of CAMP is not conserved in the murine system. In FIG. 14A, total RNA from bone marrow cells flushed from the femurs of either C/EBPε or VDR wild type (WT) or knockout (KO) mice were analyzed for murine CAMP (CRAMP) expression by Northern blot (left panel). Total RNA of BM cells from BNX mice treated for six weeks either with vehicle (−), 1,25[OH]₂D₃ (D₃) or vitamin D₃ analog compound I were examined for CRAMP expression (middle panel). The murine myeloid cell line 32Dcl3 was treated either with vehicle (0) or 1,25[OH]₂D₃ at 1×10⁻⁷ M for 24 and 48 h. Total RNA was analyzed by Northern blot for CRAMP expression (right panel). The levels of β-actin were used to demonstrate even loading of the samples. In FIG. 14B, the BM cells from C/EBPε WT or KO mice were cultured either with vehicle (−) or 1,25[OH]₂D₃ for 24 h. Relative levels of CRAMP were determined by QRT-PCR. In FIG. 14C, BM Mφ from VDR WT or KO mice were treated either with vehicle (−) or 1,25[OH]₂D₃ for 24 or 48 h. Relative levels for CRAMP were determined by QRT-PCR. FIG. 14D depicts a screen shot (Human May 2004 Assembly) of the human CAMP genomic region (chr3: 48, 237, 952-48, 423, 990; UCSC Genome Browser) (Kent, W. J. et al., “The human genome browser at UCSC,” Genome Res, Vol. 12, pp. 996-1006 (2002); Karolchik, D. et al., “The UCSC Genome Browser Database,” Nucleic Acids Res, Vol. 31, pp. 51-54 (2003)). The conservation of the human genome (International Human Genome Sequencing Consortium) compared with the chimpanzee (Chimpanzee Genome Sequencing Consortium), dog (The Broad Institute and Agencourt Bioscience), rat (Rat Genome Sequencing Consortium) and mouse (Mouse Genome Sequencing Consortium) genomes are depicted by the histograms and the Alignment Net. The positions of SINEs and LINEs are indicated. The location of the VDRE within the SINE is indicated by the arrow.

FIG. 15: Biological functions of hCAP18. hCAP18 and its murine homolog CRAMP display numerous biological functions. These include protection from bacterial infection (bactericidal activity, promoting opsonization, binding endotoxin to protect against sepsis), regulation of inflammation (chemoattractant for inflammatory cells, activating release of pro- and anti-inflammatory molecules), promotion of wound healing, and promotion of angiogenesis.

### DETAILED DESCRIPTION

The embodiments discussed herein identify extracellular signals and the downstream transcription factors that activate the transcription of the CAMP gene with a goal of, for example, extrinsically manipulating its endogenous expression for systemic and localized therapeutic benefit. This resulted in evidence that the CAMP gene is a direct target of the transcription factor vitamin D receptor (VDR) that mediates the strong upregulation of CAMP in response to treatment of cells with 1,25-dihydroxyvitamin D₃ [1,25(OH)₂D₃ or vitamin D₃] and its analogs. Induction of the endogenous CAMP by these relatively safe (FDA approved) compounds may provide important novel therapeutic uses from promotion of wound healing to protection against bacteremia and sepsis after surgery, chemotherapy or severe burns, as well as indications in the treatment of skin infections and infections of the colon. Still other indications will be readily recognized by one of skill in the art and are incorporated in various embodiments.

Thus, the present invention is based on the surprising discovery that 1,25-dihydroxyvitamin D₃ and its analogs induced expression of the human cathelicidin antimicrobial peptide (CAMP) gene. This induction was observed in acute myeloid leukemia (AML), immortalized keratinocyte and colon cancer cell lines, as well as normal human bone marrow (BM)-derived macrophages and fresh BM cells from two normal individuals and one AML patient. The induction occurred via a consensus vitamin D response element (VDRE) in the CAMP promoter that was bound by the vitamin D receptor (VDR). Induction of CAMP in murine cells was not observed and expression of CAMP mRNA in murine VDR-deficient bone marrow was similar to wild type levels. Comparison of mammalian genomes revealed evolutionary conservation of the VDRE in a short interspersed nuclear element, or SINE, in the CAMP promoter of primates that was absent in the mouse, rat or canine genomes. These findings reveal a novel activity of 1,25-dihydroxyvitamin D₃ and the VDR in regulation of primate innate immunity. Further, a recent report was consistent with the findings disclosed herein (Wang, T. T. et al., “Cutting edge: 1,25-dihydroxyvitamin D3 is a direct inducer of antimicrobial peptide gene expression,” J Immunol, Vol. 173, pp. 2909-2912 (2004)). Thus, based on the evolutionary conservation of VDREs and VDRs and/or presense of various cathelicidins or defensins, in various embodiments of the invention, Vitamin D₃ and its analogs may be used in the treatment of any mammal. “Mammal” as used herein refers to any member of the class Mammalia, including, without limitation, humans and nonhuman primates such as chimpanzees, gorillas, orangutans, capuchins, spider monkeys, marmosets and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A recent study observed induction of both CAMP and defB2 genes by 1,25(OH)₂D₃ in purified monocytes, neutrophils and cell lines from lung as well as head and neck squamous cell carcinomas (Wang, T. T. (2004)). In the current state of the art, cationic antimicrobial proteins such as cathelicidins are chemically synthesized and purified in vitro, followed by administration to a subject. The present invention is based on the discovery that Vitamin D₃ [1,25(OH)₂D₃] and its analogs strongly induce expression of hCAP18 mRNA by the CAMP gene. This invention provides an advantage over the prior art in that it provides a means for a cathelicidin and/or a defensin to be synthesized endogenously by administering Vitamin D₃, Vitamin D₃ analogs, or a combination thereof to a subject. Furthermore, in various embodiments there is the added advantage of utilizing compounds (Vitamin D₃ and Vitamin D₃ analogs) that have already been approved for use in humans and in agricultural and veterinary applications. Increased levels of cathelicidins and/or defensins may be used to treat bacterial infection, sepsis, or wounds, increase angiogenesis, modulate inflammation, and increase the efficacy of keratinocyte grafts by combating infection in contaminated wounds.

The various embodiments discussed herein expand on the prior art observations by demonstrating that 1,25(OH)₂D₃ and its analogs induce CAMP gene expression. This induction was shown to occur in the cells of the bone marrow. Moreover, the induction of CAMP by vitamin D₃ does not occur in mice. However, in various embodiments, Vitamin D₃, Vitamin D₃ analogs, or combinations thereof may be directed at other cathelicidins and/or defensins. These applications will be readily recognized by one of skill in the art and are incorporated in various embodiments. While not wishing to be bound by any theory, it appears that the mechanism for CAMP induction is conserved in primates (humans and chimpanzees) and not in other mammals as suggested by the absence of the VDRE in the murine, rat, and canine CAMP promoters. The VDRE is present in a SINE element of the Alu-Sx subfamily. These elements can retrotranspose from a progenitor element to other locations in the genome during evolution, and it would appear that this event occurred in a primate progenitor.

While these observations further expand the role of vitamin D₃ in immunomodulation in humans (Hayes, C. E. et al., “The immunological functions of the vitamin D endocrine system,” Cell Mol Biol (Noisy-le-grand) JID—9216789, Vol. 49, pp. 277-300 (2003); White, J. H., “Profiling 1,25-dihydroxyvitamin D3-regulated gene expression by microarray analysis,” J Steroid Biochem Mol Biol, Vol. 89-90, pp. 239-244 (2004)), they also indicate that the use of vitamin D₃ and its analogs provides a method to manipulate extrinsically the expression of CAMP and/or defensins; thus, relatively safe compounds can be used in the treatment of human disease and injury. Furthermore, the use of vitamin D₃ and its analogs in various agricultural and veterinary applications through the use of these compounds is also provided. For example, in various embodiments, vitamin D₃ and its analogs may be used in any mammal to increase levels of cathelicidins and/or defensins to treat bacterial infection, sepsis, or wounds, increase angiogenesis, modulate inflammation, and increase the efficacy of keratinocyte grafts by combating infection in contaminated wounds.

Increasing CAMP expression by vitamin D₃ treatment may prove beneficial in other instances. CAMP is upregulated in gastric inflammation caused by Heliobacter pylon infection (Hase, K. et al., “Expression of LL-37 by human gastric epithelial cells as a potential host defense mechanism against Helicobacter pylori,” Gastroenterology, Vol. 125, pp. 1613-1625 (2003)) and infection of cultured epithelial cells with Salmonella and entero-invasive Escherichia coli modestly induced CAMP mRNA expression (Hase, K. et al., “Cell differentiation is a key determinant of cathelicidin LL-37/human cationic antimicrobial protein 18 expression by human colon epithelium,” Infect Immun, Vol. 70, pp. 953-963 (2002). In contrast, infection by Shigella spp. was reported to downregulate CAMP mRNA expression in the colon (Islam, D. et al., “Downregulation of bactericidal peptides in enteric infections: a novel immune escape mechanism with bacterial DNA as a potential regulator,” Nat Med, Vol. 7, pp. 180-185 (2001)). Chronic oral bacterial infections occur in Kostmann syndrome patients who suffer from a severe chronic neutropenia. These patients lack expression of hCAP18 in their saliva, plasma and neutrophils (Putsep, K. (2002)). Patients suffering from specific granule deficiency (SGD), lack expression of both defensins and hCAP18 and suffer severe, recurrent bacterial infections (Gombart, A. F. et al., “Neutrophil specific granule deficiency and mutations in the gene encoding transcription factor C/EBP(epsilon),” Curr Opin Hematol, Vol. 9, pp. 36-42 (2002)). Further, decreased levels of hCAP18 in the skin of individuals with atopic dermatitis (AD) correlates with their increased susceptibility to skin infection as compared to those with psoriasis (Ong, P. Y. (2002)). Upregulating CAMP/hCAP18 expression in these conditions could prove therapeutically beneficial.

The induction of CAMP expression by cytokines and growth factors has been reported in a number of tissues; but 1,25(OH)₂D₃ and its analogs are strikingly potent in myeloid cells. The induction was less striking in the HaCat and HT-29 cell lines; but combining vitamin D₃ treatment with other compounds known to activate CAMP expression may increase expression. For example, treatment of cultured keratinocytes or composite keratinocyte grafts with LPS or IL-1β induced CAMP expression (Erdag, G. et al., “Interleukin-1alpha and interleukin-6 enhance the antibacterial properties of cultured composite keratinocyte grafts,” Ann Surg, Vol. 235, pp. 113-124 (2002)). On the other hand, TNFα, II-4, II-6, IL-8, IL-10 and INFγ did not. The growth factor insulin-like growth factor (IGF)-1 that is important in wound healing was found to induce both the CAMP mRNA and protein in primary human keratinocytes, but TGFα and proinflammatory cytokines IL-1β, IL-6 and TNFα were not (Sorensen, O. E. et al., “Wound healing and expression of antimicrobial peptides/polypeptides in human keratinocytes, a consequence of common growth factors,” J Immunol, Vol. 170, pp. 5583-5589 (2003)). In epithelial cells of the colon, hCAP18 expression is restricted to differentiated cells in the human colon and ileum (Hase, K. (2002); Schauber, J. et al., “Expression of the cathelicidin LL-37 is modulated by short chain fatty acids in colonocytes: relevance of signalling pathways,” Gut, Vol. 52, pp. 735-741 (2003)). Consistent with this, hCAP18 expression was induced by differentiation of colon epithelial cell lines and by short chain fatty acids independent of differentiation, but not by pro-inflammatory mediators including IL-1α, IL-6, TNFα, INFγ, LPS or PMA (Hase, K. (2002); Schauber, J. (2003)). Combining those cytokines or growth factors with vitamin D₃ offers the possibility of obtaining synergistic activation of the CAMP gene. Such synergy was reported for LPS and vitamin D₃ in neutrophils (Wang, T. T. (2004)). Synergistic activation of the CAMP gene could prove useful in treating skin grafts for burn patients or in boosting immunity to opportunistic infections in chemotherapy patients.

In various embodiments, the present invention is directed to compositions and methods involving cationic antimicrobial peptides. In other embodiments, the exemplary aspects discussed herein relate to vitamin D and vitamin D analogues. Various embodiments also relate to their use in pharmaceutical compositions intended for use in human or veterinary medicine, or alternatively in cosmetic compositions or agricultural compositions.

The cationic antimicrobial peptides, vitamin D and vitamin D analogues according to the invention have pronounced activity in the fields of cell proliferation and differentiation and find applications in the treatment of microbial infections, skin infections and infections of the colon, sepsis, promotion of wound healing, angiogenesis or chemoattraction. Other applications will be recognized by one of skill in the art, including, but not limited to, the topical and systemic treatment of dermatological (or other) ailments associated with a keratinization disorder, ailments with an inflammatory and/or immunoallergic component and hyperproliferation of tissues of ectodermal origin (skin, epithelium, etc.), whether benign or malignant. These compounds can also be used to combat aging of the skin, whether light-induced or chronological, and to treat cicatrization disorders.

Vitamin D and its analogues have properties of transactivation of the vitamin D response elements (VDRE), such as an agonist or antagonist activity with respect to receptors of vitamin D or analogs thereof. Vitamin D and Vitamin D analogs include, for example, the analogs of vitamin D₂ or D₃ and in particular 1,25-dihydroxyvitamin D₃ (calcitriol).

This agonist activity with respect to receptors of vitamin D or analogs thereof can be demonstrated “in vitro” by methods known in the field of study of gene transcription (Hansen et al., The Society for Investigative Dermatologie, vol. 1, No. 1, April 1996).

By way of example, the VDR agonist activity can be tested on the HeLa cell line by co-transfection with an expression vector for the human VDR receptor and the reporter plasmid p240Hase-CAT which contains the region −1399 to +76 of rat 24-hydroxylase promoter, cloned upstream of the frame encoding the chloramphenicol-acetyl-transferase (CAT)′gene. Eighteen hours after co-transfection, the test product is added to the medium. Eighteen hours after treatment, assay of the CAT activity in the cell lysates is carried out by an ELISA test. The results are expressed as percentages of the effect normally observed with 10⁻⁷ M calcitriol. The agonist activity can also be characterized in this co-transfection system, by determining the dose required to reach 50% of the maximum activity of the product.

The biological properties of the vitamin D analogues can also be measured by the capacity of the product to inhibit the proliferation of normal human keratinocytes (NHK in culture). The product is added to NHKs cultured under conditions which promote the proliferative state. The product is left in contact with the cells for 5 days. The number of proliferative cells is measured by incorporation of bromodeoxyuridine (BRdU) into the DNA.

The vitamin D receptor agonist activity of the compounds of the invention can also be evaluated “in vivo” by induction of 24-hydroxylase in SKH mice. (Voorhees et al., 1997.108: 513-518).

The compounds according to the invention may be suitable for use in the following fields of treatment. For example, the compounds may be suitable in any instance where altering the expression of any cathelicidin and/or defensin or CAMP, hCAP18 and/or defB2 may have a beneficial effect in treating a condition. A “beneficial effect, as used herein may include, but is in no way limited to, lessening the severity of the condition, preventing the condition from worsening, curing the condition and prolonging a patient's life or life expectancy. “Treatment” and “treating,” as used herein include preventing, inhibiting, curing, and alleviating the conditions or symptoms thereof. “Conditions” as used herein include a wide range of physiological issues. For instance, the compounds may be useful in treating dermatological ailments associated with a keratinization disorder which has a bearing on differentiation and on proliferation, in particular for treating simple acne, comedones, polymorphonuclear leukocytes, rosacea, nodulocystic acne, acne conglobata, senile acne and secondary acne such as solar, medication-related or professional acne. They may be useful for treating other types of keratinization disorders, in particular ichthyosis, ichthyosiform states, Darier's disease, palmoplantar keratoderma, leukoplasias and leukoplasiform states, and cutaneous or mucous (buccal) lichen. They may be useful for treating other dermatological ailments with an inflammatory immunoallergic component, with or without cell proliferation disorder, and, in particular, all forms of psoriasis, whether this is cutaneous, mucous or ungual psoriasis, and even psoriatic rheumatism, or alternatively cutaneous atopy, such as eczema or respiratory atopy or alternatively gingival hypertrophy. They may be useful for treating all dermal or epidermal proliferations, whether benign or malignant and whether they are of viral origin or otherwise, such as common warts, flat warts and verruciform epidermodysplasia, oral or florid papillomatoses, T lymphoma and proliferations which may be induced by ultraviolet radiation, in particular in the case of basocellular and spinocellular epithelioma, as well as any pre-cancerous skin lesion such as keratoacanthomas. They may be useful for treating other dermatological disorders such as immune dermatitis such as lupus erythematosus, immune bullosis and collagen diseases such as scleroderma. They may be useful in the treatment of dermatological or general ailments with an immunological component, for combating disorders of sebaceous function such as the hyperseborrhoea of acne or simple seborrhoea, for the treatment of skin disorders due to exposure to UV radiation, as well as for repairing or combating ageing of the skin, whether it is light-induced or chronological ageing, or for reducing actinic keratoses and pigmentations, or any pathologies associated with chronological or actinic ageing. They may be useful for preventing or treating cicatrization disorders or for preventing or repairing stretchmarks, for the treatment of inflammatory ailments such as arthritis, for the treatment of any complaint of viral origin on the skin or generally, such as Kaposi's syndrome. They may be useful for treating certain ophthalmological disorders, in particular corneopathies, for the treatment or prevention of cancerous or pre-cancerous states of cancers presenting or possibly being induced by vitamin D receptors, such as, but without limitation, breast cancer, leukaemia, myelodysplasic syndromes and lymphomas, carcinomas of the Malpighian epithelial cells and gastrointestinal cancers, melanomas and osteosarcoma. They may be useful in the prevention or treatment of alopecia of various origins, in particular alopecia due to chemotherapy or radiation. They may be useful for the treatment of immune ailments, such as autoimmune diseases, for instance type 1 diabetes mellitus, multiple sclerosis, lupus and lupus-type ailments, asthma, glomerulonephritis, selective dysfunctions of the immune system such as AIDS, or prevention of immune rejection such as kidney, heart, bone marrow, liver, pancreatic islets, pancreas or skin graft rejects, or prevention of graft-versus-host disease. They may be useful for the treatment of endocrine ailments, given that the vitamin D analogues can modify hormonal secretion such as increasing the secretion of insulin or selectively suppressing the secretion of parathyroid hormone, for example in chronic renal insufficiency and secondary hyperparathyroidism. They may be useful for the treatment of ailments characterized by abnormal management of intracellular calcium, and in the treatment or prevention of pathologies in which calcium metabolism is involved, such as muscular ischaemia (myocardial infarction). They may be useful for treatment or prevention of vitamin D deficiencies and other mineral homeostasis ailments in plasma and bone, such as rickets, osteomalacia, osteoporosis, in particular in the case of menopausal women, renal osteodystrophy and parathyroid function disorders. And they may be useful in the treatment of ailments of the cardiovascular system such as arteriosclerosis or hypertension; as well as non-insulin-dependent diabetes. Still other indications will be readily recognized by one of skill in the art and are incorporated in various embodiments.

In the therapeutic indications discussed, supra, in various embodiments, the compounds can advantageously be used in combination with other therapeutic indications known to one of skill in the art. For example, cationic antimicrobial peptides, vitamin D and vitamin D analogues according to the invention may be administered with any number of established therapeutics including, but in no way limited to, retinoids, with corticosteroids or oestrogens, in combination with antioxidants, with α-hydroxy or α-keto acids or derivatives thereof, with potassium-channel blockers, or alternatively in combination with other medicinal products known to interfere with the immune system (for example cyclosporin, FK 506, glucocorticoids, monoclonal antibodies, cytokines or growth factors, etc.).

In various embodiments, the cationic antimicrobial peptides, vitamin D and vitamin D analogues according to the invention may also be included in a pharmaceutical composition. Thus, also included are pharmaceutical compositions intended for treating the above-mentioned ailments.

The cationic antimicrobial peptides, vitamin D and vitamin D analogues according to the invention can be administered via any route of administration. “Route of administration” may refer to any administration pathway known in the art, including but not limited to aerosol, enteral, nasal, ophthalmic, oral, parenteral, rectal, transdermal (e.g., topical cream or ointment, patch), or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal.

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain inert or even pharmacodynamically or cosmetically active additives or combinations of these additives, including, but in no way limited to: wetting agents; depigmenting agents such as hydroquinone, azelaic acid, caffeic acid or kojic acid; emollients; moisturizing agents such as glycerol, PEG 400, thiamorpholinone and derivatives thereof or urea; antiseborrhoeic or antiacne agents, such as S-carboxymethylcysteine or S-benzylcysteamine and salts and derivatives thereof, or benzoyl peroxide; antibiotics such as erythromycin and esters thereof, neomycin, clindamycin and esters thereof, tetracyclines; antifungal agents such as ketoconazole or poly-4,5-methylene-3-isothiazolinones; agents for promoting regrowth of the hair, such as Minoxidil (2,4-diamino-6-piperidinopyrimidine 3-oxide) and derivatives thereof, Diazoxide (7-chloro-3-methyl-1,2,4-benzothiadiazine 1,1-dioxide) and Phenytoin (5,4-diphenylimidazolidine-2,4-dione); non-steroidal anti-inflammatory agents; carotenoids, and in particular β-carotene; anti-psoriatic agents such as anthralin and derivatives thereof, and, eicosa-5,8,11,14-tetraynoic acid and eicosa-5,8,11-triynoic acid, and esters and amides thereof.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs that it may come in contact with, meaning that it must not carry a risk of toxicity, irritation, allergic response, immunogenecity, or any other complication that excessively outweighs its therapeutic benefits.

The compositions according to the invention can also contain flavour enhancers, preserving agents such as para-hydroxybenzoic acid esters, stabilizers, moisture regulators, pH regulators, osmotic pressure modifiers, emulsifiers, UV-A and UV-B screening agents, antioxidants such as α-tocopherol, butylhydroxyanisole or butylhydroxytoluene.

The pharmaceutical compositions according to the invention may be delivered in a therapeutically effective amount. “Therapeutically effective amount” as used herein refers to an amount of a compound that produces a desired therapeutic effect, such as preventing or treating a target condition or alleviating symptoms associated with the condition. Specifically, in one embodiment, a “therapeutically effective amount” in the present invention is that amount of Vitamin D₃, one or more Vitamin D₃ analogs, or both Vitamin D₃ and one or more Vitamin D₃ analogs necessary to induce cathelicidin production sufficient to treat sepsis, a microbial infection, or a wound in a subject. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20^{th} edition, Williams & Wilkins PA, USA) (2000).

“Vitamin D₃ analog” refers to any structural analog of Vitamin D₃. Examples of suitable Vitamin D₃ analogs include but are not limited to calcipotriol (also known as calcipotriene, or MC903) (Calverley 1987), maxacalcitol (22-oxy-1α,25(OH)₂D₃, also known as 22-oxacalcitriol or OCT) (Abe 1987), paricalcitol (19-nor-1,25-(OH)₂D₂) (Takahashi 1987), tacalcitol (1α,24(R)-(OH)₂D₃) (Shimura 1979), doxercalciferol (1α-OH-D₂) (Frazao 1998), alfacalcidol (1α-OH-D₃), SM-10193 (F6-1,23(S),25-(OH)₃D₃), lexacalcitol (KH1060) (Dilworth 1997), seocalcitol (EB1089) (U.S. Pat. No. 5,190,935), EB1072 (Quack 1998), EB1129 (Quack 1998), EB1133 (Quack 1998), EB1155 (Quack 1998), EB1270 (Quack 1989), MC1288 (Vaisanen 1999), EB1213 (Bury 2001), CB1093 (Danielsson 1997), CB966 (Vink-van Wijngaarden 1994), VD2656 (Vaisanen 1999), VD2668 (Vaisanen 1999), VD2708 (Vaisanen 1999), VD2716 (Vaisanen 1999), VD2728 (Vaisanen 1999), VD2736 (Vaisanen 1999), GS1500 (Vaisanen 1999), GS1558 (Vaisanen 1999), KH1060 (Vink-van Wijngaarden 1994), ZK161422 (Herdick 2000), and Vitamin D₃ analog I (1,25R,26-(OH)₂-22-ene-D₃) (Bouillon 1995). Other Vitamin D₃ analogs will be readily recognized by one of skill in the art. See, e.g. U.S. Pat. No. 6,831,106; U.S. Pat. No. 6,706,725; and U.S. Pat. No. 6,689,922; Posner, G. H., “Low-Calcemic Vitamin D Analogs (Deltanoids) for Human Cancer Prevention,” J. Nutr., Vol. 132, pp. 3802S-3803S (2002); Bouillon, R. et al., “Structure-Function Relationships in the Vitamin D Endocrine System,” Endocrine Reviews, Vol. 16, No. 2, pp. 200-257 (1995); Qiao, G. et al., “Analogs of 1α,25-dihydroxyvitamin D3 as novel inhibitors of renin biosynthesis,” J Steriod Biochem Mol Biol. (May 5, 2005).

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

### Example 1

### Tissue Culture and Reporter Assays

The human myeloid leukemia cell lines U937, NB4, HL60 and ML1 were cultured in RPMI1640 (obtained from Invitrogen Corp.; Carlsbad, Calif.) containing 10% fetal calf serum (FCS) (obtained from Omega Scientific, Inc.; Tarzana, Calif.). The human bone marrow cells isolated either from two normal or one acute myeloid leukemia patient were cultured in RPMI1640 containing 10% FCS for short-term experiments. Bone marrow (BM)-derived macrophages (Mφ) were obtained by culturing normal human bone marrow (NHBM) cells in RPMI1640 containing 10% FCS, 200 ng/ml GM-CSF and 5% WeHi-3B conditioned medium (source of IL-3) for 14 days. The bone marrow samples were obtained from patients after informed consent was given. The immortalized keratinocyte cell line, HaCat (a kind gift from Dr. Norbert Fusenig, Heidelberg, Germany), and the colon cancer cell line, HT29, were cultured in DMEM containing 10% FCS. All media were supplemented with antibiotics (100 units penicillin/streptomycin) (obtained from Invitrogen).

Cells were treated with various concentrations and durations of 1,25(OH)₂D₃, a vitamin D₃ analog or vehicle (ethanol). The 1,25(OH)₂D₃ and Compound I (1,25R,26-(OH)₃-22-ene-D₃) were synthesized and generously provided by Dr. Milan Uskokovic at Hoffmann-LaRoche, Inc. (Nutley, N.J.). The analogs KH1060 (20-epi-22oxa-24a,26a,27a-tri-homo-1,25(OH)2D3) and EB1089 (1,25-dihydroxy-22,24-diene, 24,26,27-trihomo) were synthesized by Leo Pharmaceutical Products (Ballerup, Denmark) and generously provided by Dr. Lise Binderup. U937 cells were treated for 24 h either with vehicle (ethanol), LPS (1 μg/ml), TPA (10 ng/ml), TNFα (1 ng/ml), INFα (10 ng/ml), IFNγ (50 ng/ml), IL-2 (2.5 ng/ml), IL-6 (10 ng/ml), GM-CSF (1 ng/ml), G-CSF (60 ng/ml), estradiol (1×10⁻⁸ M), dihydrotestosterone (DHT, 1×10⁻⁸ M) or all trans retinoic acid (ATRA, 5×10⁻⁷ M). Cyclohexamide (obtained from Sigma; St. Louis, Mo.) was used at 20 μg/ml and the absence of protein synthesis was determined by measuring ³⁵S-methionine incorporation. Cyclohexaminde was added 30 min prior to adding the vehicle or 1,25(OH)₂D₃. Actinomycin D (obtained from Sigma) was used at 10 μg/ml and was added at the same time as vehicle or 1,25(OH)₂D₃.

Murine 32Dcl3 cells provided by Alan Friedman (Johns Hopkins University; Baltimore, Md.) were cultured in IMDM (obtained from Invitrogen) supplemented with 10% FCS and 10% Wehi3B-conditioned medium. Cells were treated with 1,25-dihydroxyvitamin D3 or ethanol for 0, 24 and 48 h and total RNA harvested. The 1,25(OH)₂D₃ and compound I (both 0.05 pg/mouse) were administered to beige/nude/x-linked (bnx) nu/nu nude mice every two days for 6 weeks. The bone marrow cells were flushed from the femurs and total RNA isolated. Bone marrow cells were flushed from femurs of either VDR-deficient mice or wild type littermates (Yoshizawa, T. et al., “Mice lacking the vitamin D receptor exhibit impaired bone formation, uterine hypoplasia and growth retardation after weaning,” Nat Genet, Vol. 16, pp. 391-396 (1997)). Red blood cells were lysed and cells plated in IMDM supplemented with 10% FCS. Cells were treated with 1,25(OH)₂D₃ or ethanol for 24 h and total RNA harvested. BM-derived macrophages were obtained from VDR-deficient and wild type murine femurs as described previously (Tavor, S. et al., “Macrophage functional maturation and cytokine production are impaired in C/EBP epsilon-deficient mice,” Blood, Vol. 99, pp. 1794-1801 (2002)). Cells were treated with ethanol or 1,25(OH)₂D₃ for 0, 24 and 48 h and total RNA harvested.

U937 cells were electroporated using a BTX T820 (obtained from Genetronics Biomedical, Ltd.; San Diego, Calif.). The settings were low voltage, 200 V, 10 msec, 1 pulse in 250 μl of cells at 2×10⁷ cells/ml in a 4 mm cuvette. A total of 20 μg plasmid was used per transfection. After transfection, cells were treated with either 1,25(OH)₂D₃ or vehicle. Cell lysates were prepared and luciferase activities determined using the dual luciferase assay system as described by the manufacturer (obtained from Promega Corp.; Madison, Wis.). Transfection efficiency was normalized to the renilla luciferase expression vector phTKRL (obtained from Promega Corporation).

### Example 2

### Construction of Recombinant Plasmids

Primers (SEQ ID Nos. 2 and 3) (Table 1) were used to amplify the human CAMP promoter (nucleotides −693 to +14) from human genomic DNA (Larrick, J. W. et al., “Structural, functional analysis and localization of the human CAP18 gene,” FEBS Lett, Vol. 398, pp.74-80 (1996)).

TABLE 1

| | |
|---|---|
| SEQ ID NO.:2 | SEQ ID NO.:3 |

| | |
|---|---|
| 5′-CCGACGCGTCATACTGAGTC | 5′-CCGCTCGAGGGTCCCCATGTCTG |
| TCACTCTGTTACC-3′ | CCTC-3′ |

This fragment was subcloned into the firefly luciferase reporter plasmid pXP2 (Nordeen, S. K., “Luciferase reporter gene vectors for analysis of promoters and enhancers,” Biotechniques, Vol. 6, pp. 454-458 (1998)) and called pXP2-CAMP-Luc. Subsequently deletion mutants pXP2-CAMP(ΔSmaI)-Luc and pXP2-CAMP(ΔHindIII)-Luc were generated by restriction enzyme digestion, fill-in and religation of the purified linear plasmid. Constructs were verified by nucleotide sequencing.

### Example 3

### Analysis of RNA and Protein Expression

Total RNA was prepared using Trizol Reagent (obtained from Invitrogen), electrophoresed through a formaldehyde-containing, 1% agarose gel and transferred to a positively charged nylon membrane (Hybond N+) for Northern analysis (obtained from Amersham Pharmacia Biotech; Piscataway, N.J.). The blots were sequentially probed with ³²P-labeled DNA probes (Strip-EZ™; obtained from Ambion, Inc.; Austin, Tex.) specific for the CAMP, CDIIb and β-actin mRNAs.

For quantitative real-time PCR (QRT-PCR), total RNA was prepared, treated with DnaseI (obtained from Invitrogen) and cDNAs were synthesized by reverse transcription using Superscript II reverse transcriptase as described by the manufacturer (obtained from Invitrogen). The cDNAs were then analyzed by QRT-PCR using a fluorescent probe (obtained from Applied Biosystems; Foster City, Calif.) against either CAMP (5′-6fam-[SEQ ID NO.: 4]-tamra-3′) (Table 2) or 18S (Tsukasaki, K. et al., “Identifying progression-associated genes in adult T-cell leukemia/lymphoma by using oligonucleotide microarrays,” Int J Cancer, Vol. 109, pp. 875-881 (2004)) at a final concentration of 200 nM per reaction. Primers against CAMP (forward, SEQ ID NO.: 5 and reverse, SEQ ID NO.: 6) (Table 2) or 18S (Tsukasaki, K. (2004)) were used at 600 nM per reaction.

TABLE 2

| | | |
|---|---|---|
| SEQ ID NO.:4 | SEQ ID NO.:5 | SEQ ID NO.:6 |

| | | |
|---|---|---|
| 5′-6fam-[ACCCCAGGCC | 5′-GCTAACCTCT | 5′-GGTCACTGTC |
| CCACGATGGAT]-tamra-3′ | ACCGCCTCCT-3′ | CCCATACACC-3′ |

PCR was performed using HotMaster™ Taq polymerase (obtained from Eppendorf AG; Hamburg, Germany) on an iCycler PCR machine equipped with an optical module (obtained from Bio-Rad Laboratories; Hercules, Calif.). The protocol was 95° C., 1 min followed by 45 cycles of 95° C., 15 sec and 60° C., 1 min during which time data collection occurred. Standard curves were generated by PCR using serial dilutions of known quantities of CAMP or 18S cDNA and were included on each plate to quantify either the pg of CAMP or ng of 18S cDNA in each sample. PCR was performed in triplicate for each sample.

Primers against murine CAMP/CRAMP (forward, SEQ ID NO.: 7 and reverse, SEQ ID NO.: 8) (Table 3) were used at 200 nM per reaction.

TABLE 3

| | |
|---|---|
| SEQ ID NO.:7 | SEQ ID NO.:8 |

| | |
|---|---|
| 5′-GCAGTTCCAG | 5′-GTTCCTTGAAGGCACATTGC-3′ |
| AGGGACGTC-3′ | |

PCR was performed using SYBR green (obtained from Molecular Probes, Eugene, Oreg.) as previously described (Tsukasaki, K. (2004)). The protocol was 95° C., 1 min followed by 45 cycles of 95° C., 15 sec; 60° C., 30 sec and 65° C., 1 min during which time data was collected. The relative fold change between samples was determined using data normalized for 18S expression. Samples were analyzed in triplicate.

Western blot and immunfluorescent microscopy analyses were essentially performed as previously described (Gombart, A. F. et al., “Mutations in the gene encoding the transcription factor CCAAT/enhancer binding protein alpha in myelodysplastic syndromes and acute myeloid leukemias,” Blood, Vol. 99, pp. 1332-1340 (2002)). The total cell lysates were electrophoresed through 20% polyacrylamide-SDS gels. The hCAP18 antibody was used at 2.0 μg/ml for Western blot analysis and 4.0 μg/ml for immunofluorescent (IF) microscopy (Sorensen, O. (1997)). The anti-GAPDH monoclonal antibody was used at a 1:10,000 dilution (obtained from Research Diagnostics, Inc.; Flanders, N.J.).

### Example 4

### Chromatin Immunoprecipitation (ChIP) Assays

The ChIP assays were performed essentially as described by the manufacturer (obtained from Upstate, Inc.; Chalottesville, Va.). Approximately 1×10⁷ cells were incubated with either vehicle or 1,25-dihydroxyvitamin D₃ (1×10⁻⁷ M for 4 h). Protein/DNA complexes were crosslinked in 1% formaldehyde for 10 min. The reaction was terminated with the addition of glycine to 0.125 M final concentration. The cells were washed in ice-cold PBS containing PMSF (10 μg/ml), resuspended in 1 ml of SDS-lysis buffer containing protease inhibitors and incubated on ice for 10 min. The lysates were sonicated 3×, 10 seconds at 30% output to shear the DNA. The sonicated lysate was pelleted at 13K rpm for 10 min at 4° C. Supernatant (0.2 ml) was mixed with 1.8 ml of dilution buffer and precleared with protein A-agarose for 1 h on ice. Antibody (2 μg) against VDR (mixed SC-1008 [1 μg] and SC-1009 [1 μg]) (obtained from Santa Cruz Biotechnology; Santa Cruz, Calif.), C/EBPε (2 μl) (Chumakov, A. M. et al., “Cloning of the novel human myeloid-cell-specific C/EBP-epsilon transcription factor,” Mol Cell Biol, Vol. 17, pp. 1375-86 (1997)), preimmune serum or no antibody was added and the samples incubated overnight at 4° C. A slurry of ssDNA/protein A agarose was added and the mixture incubated with rocking overnight at 4° C. The agarose/antibody/protein/DNA complex was pelleted and washed in low salt (1×), high salt (1×), LiCl (1×) and TE (2×). The complex was removed from the protein A-agarose in elution buffer (2×500 μl); crosslinks were reversed in 10 mM NaCl at 65° C. for 4 hours; proteinase K treated; phenol/chloroform extracted and ethanol precipitated. The promoter fragment was detected by PCR using primers against the CAMP promoter (forward, SEQ ID NO.: 9 and reverse, SEQ ID NO.: 10) (Table 4).

TABLE 4

| | |
|---|---|
| SEQ ID NO.:9 | SEQ ID NO.:10 |

| | |
|---|---|
| 5′-ACCGTGCCCTGCCTCATTC- | 5′-TGGTCCCCATGTCTGCCTC-3′ |
| 3′ | |

The 430-bp fragment was cloned and sequenced to verify that the CAMP promoter was amplified. QRT-PCR was performed using SYBR Green (obtained from Molecular Probes; Eugene, Oreg.) essentially as described (Tsukasaki, K. (2004)).

### Example 5

### Induction of CAMP Gene Expression by 1,25(OH)₂D₃

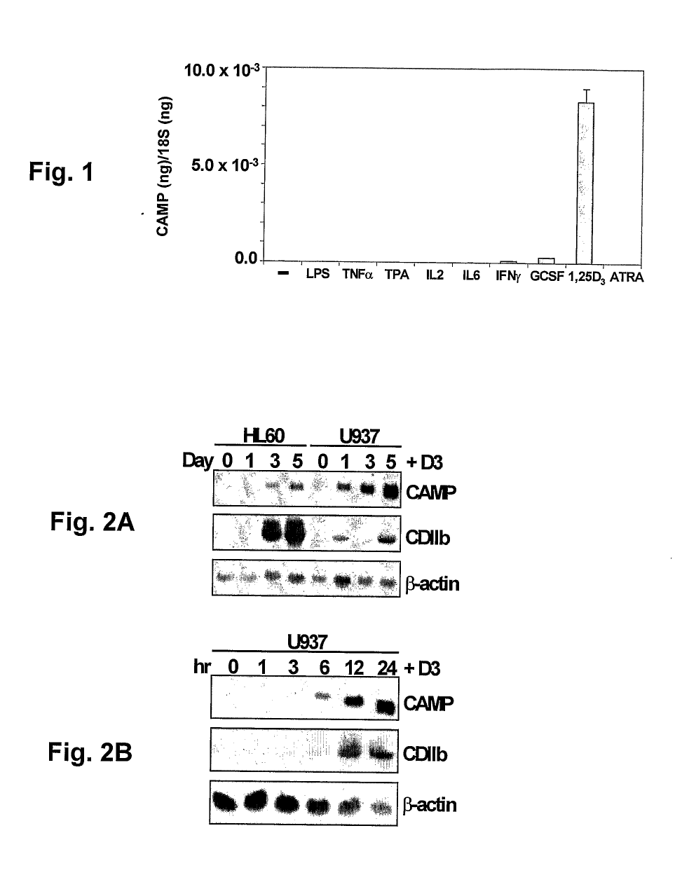

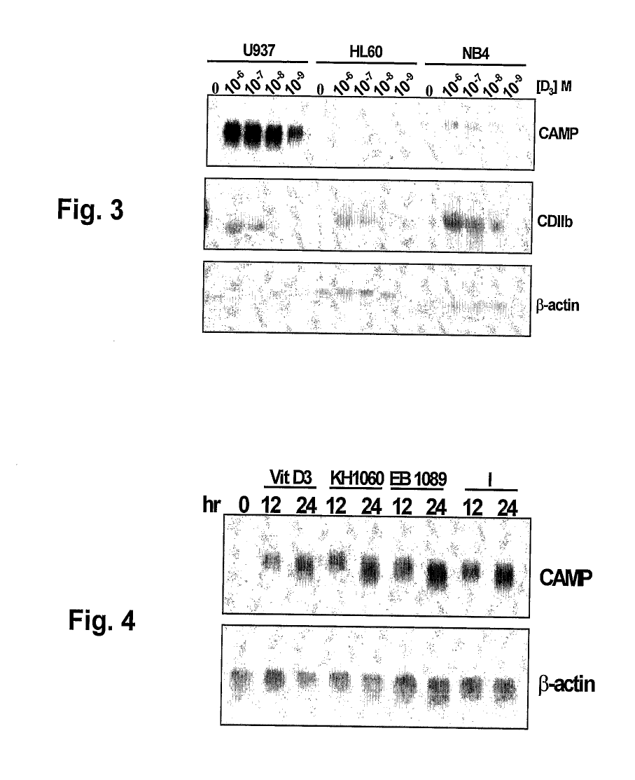

In an initial screen to identify extracellular signals that might induce CAMP gene expression, the myeloid leukemia cell line U937 was treated with various inflammatory factors (LPS, TPA, TNFα, INFα and INFγ), cytokines and growth factors (IL-2, IL-6, GM-CSF and G-CSF) and seco-steroid hormones (DHT, estradiol, ATRA and 1,25(OH)₂D₃)(FIG. 1 and data not shown). As determined by QRT-PCR and Northern blot analyses, only 1,25(OH)₂D₃ induced CAMP expression significantly (FIG. 1 and FIG. 2). Also, the induction was observed in HL60 (FIG. 2 and FIG. 3) and NB4 (FIG. 3). Induction of the CAMP gene occurred by day 1 in both the U937 and HL60 cell lines, but was stronger in the U937 cell line (FIG. 2). A time course from 1-24 hours in U937 indicated that CAMP induction began between 1-3 hours after addition of 1,25(OH)₂D₃ and prior to induction of the differentiation marker CDIIb at 12 hours (FIG. 2). CAMP induction continued throughout the five days of treatment and was dose responsive (FIG. 1 and FIG. 3). Each of the chemically synthesized 1,25(OH)₂D₃ analogs KH1060, EB1089 and compound I strongly induced CAMP gene expression (FIG. 4). Levels of induction were similar to those observed for 1,25(OH)₂D₃. No induction was observed with ATRA (5×10⁻⁷ M, 1-5 days) in U937, HL60 and NB4 (FIG. 1 and data not shown). This is consistent with the inability of human myeloid leukemia cell lines to express significant levels of mRNAs for secondary granule genes even when induced to undergo granulocytic differentiation by ATRA (Khanna-Gupta, A. et al., “NB4 cells show bilineage potential and an aberrant pattern of neutrophil secondary granule protein gene expression,” Blood, Vol. 84, pp. 294-302 (1994)).

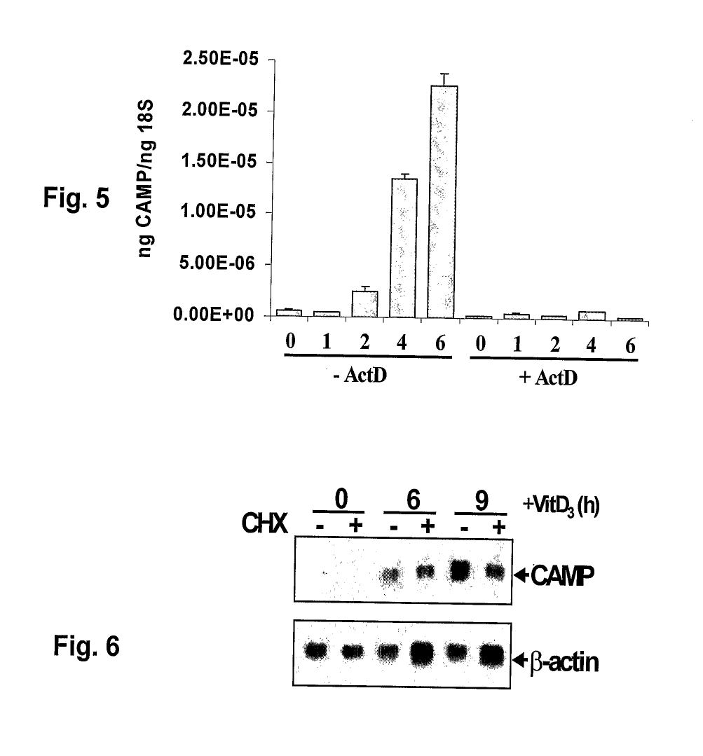

The induction of CAMP was blocked by actinomycin D indicating that it occurred at the level of transcription (FIG. 5). The data suggested that the human CAMP gene was a direct transcriptional target of the VDR. The steroid hormone receptor family members are generally present in the cytosol or bound to the DNA in an inactive state and require activation by binding ligand (Carlberg, C. et al., “Gene regulation by vitamin D3,” Crit Rev Eukaryot Gene Expr, Vol. 8, pp. 19-42 (1998)). Upon binding to ligand, they immediately translocate to the nucleus and bind vitamin D response elements (VDREs) in target genes and induce gene expression. While not wishing to be bound by any theory, this model predicts that ongoing protein synthesis is not required for this process to occur. To test this, U937 cells were treated with 1,25(OH)₂D₃ in the presence or absence of cyclohexamide (CHX) to block protein synthesis. Induction of CAMP gene expression occurred in the absence of ongoing protein synthesis (presence of CHX) (FIG. 6). CHX did not induce CAMP gene expression (data not shown). Since cyclohexamide prevents the synthesis of new proteins, these results indicate that the CAMP gene is activated by transcription factors that are already present in the cell at the time of Vitamin D₃ treatment. These data further support the hypothesis that the CAMP gene is a direct target of the VDR and not activated by secondary events such as the synthesis of other transcription factors that are induced by VDR.

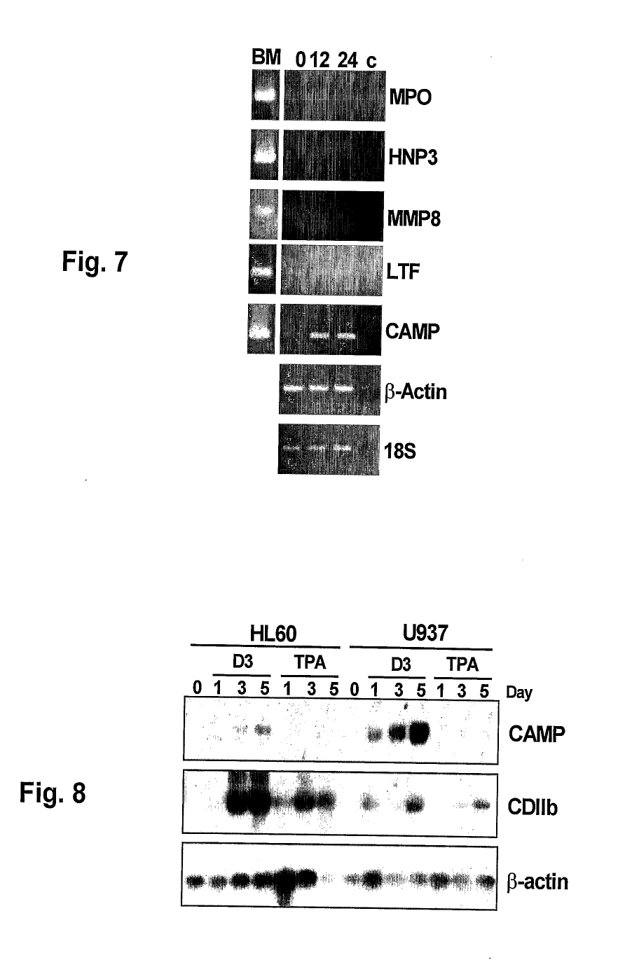

To determine the specificity of CAMP induction by 1,25(OH)₂D₃, other neutrophil primary [MPO (myeloperoxidase) and HNP3 (α-defensin)] and secondary [MMP8 (matrix metalloproteinase 8) and LTF (lactoferrin)] granule genes were tested for induction. No induction of these genes was observed after 24 h of treatment, while CAMP was significantly upregulated (FIG. 7). The data demonstrate that the 1,25(OH)₂D₃ induction of neutrophil granule genes is restricted primarily to CAMP.

### Example 6

### Induction of CAMP is Independent of Monocytic Differentiation

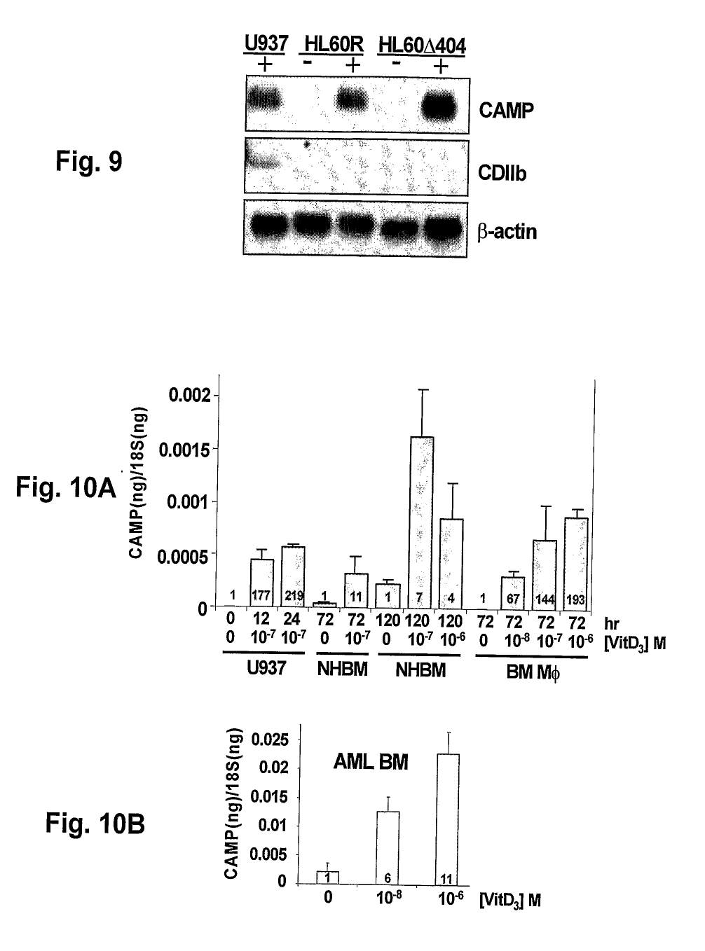

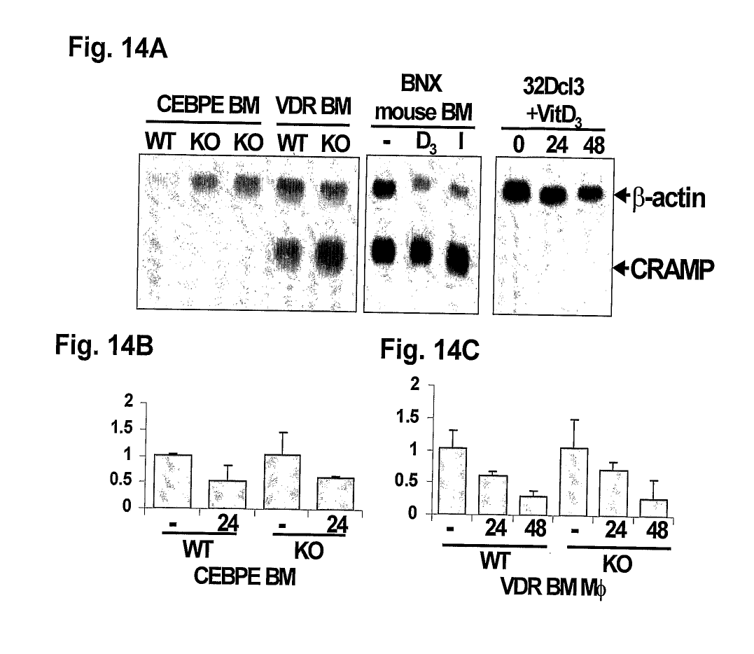

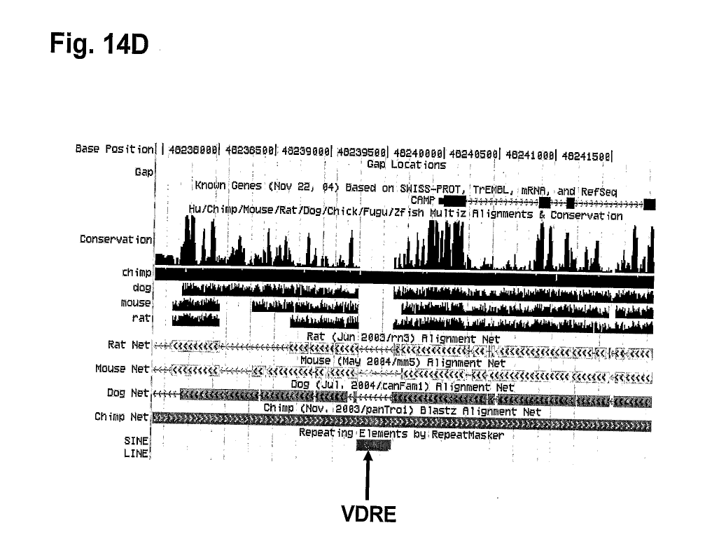

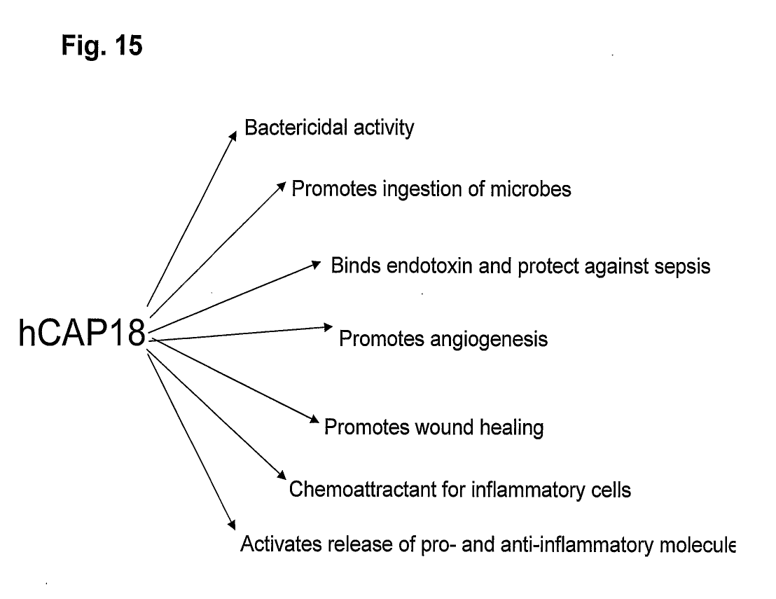

Vitamin D₃ promotes macrophage-like differentiation of U937 and HL-60 (Koeffler, H. P., “Induction of differentiation of human acute myelogenous leukemia cells: therapeutic implications,” Blood, Vol. 62, pp. 709-21 (1983)). To determine if differentiation was responsible for the induction of the CAMP gene in AML cell lines, HL-60 and U937 cells were treated either with 1,25(OH)₂D₃ or 12-O-tetradecanoylphorbol-13-acetate (TPA). TPA is known to induce differentiation in certain tumor cell lines. Both compounds promoted macrophage-like differentiation of these cells as demonstrated by the induction of the differentiation marker CDIIb, but induction of CAMP mRNA was observed only with 1,25(OH)₂D₃, not TPA (FIG. 8). The data suggested that induction of differentiation was not sufficient for CAMP expression. Furthermore, 1,25(OH)₂D₃ induced expression of CAMP was observed in the AML cell line NB4 that does not undergo macrophage differentiation when treated with 1,25(OH)₂D₃ (FIG. 3). Finally, to demonstrate that CAMP induction occurs in the absence of differentiation, sub-lines derived from HL-60 that are unable to differentiate in response to vitamin D₃ (HL60R) or ATRA (HL60Δ404) were treated with 1,25(OH)₂D₃. The HL60R cell line is unable to respond to inducers of either granulocytic or monocytic differentiation, including Vitamin D₃. This was confirmed by a lack of CDIIb expression in response to Vitamin D₃ treatment (FIG. 14B). The HL60Δ404 cell line is unable to respond to ATRA-induced granulocytic differentiation, but responds to Vitamin D₃. This was confirmed by the induction of CDIIb in response to Vitamin D₃ treatment (FIG. 14B). In both cell lines, hCAP18 expression was induced by Vitamin D₃ (FIG. 5B, upper panel). This data confirms that monocytic differentiation is not required for induction of CAMP gene expression (FIG. 9, HL60R vs. HL60Δ404, respectively).

### Example 7

### Induction of the CAMP Gene Occurs in Bone Marrow Cells from Normal Humans and a Patient Suffering from Acute Myeloid Leukemia

To determine if CAMP induction by vitamin D₃ occurs in hematopoietic cells other than leukemia cell lines, total bone marrow (BM) cells and BM-derived macrophages (BM Mφ) from two normal individuals and BM cells from one AML patient were treated with 1,25(OH)₂D₃ in vitro. Cells were cultured in RPMI 1640+10% FCS for 72 or 120 hours. Cells were treated with 1×10⁻⁸ to 1×10⁻⁶ M Vitamin D₃ at time 0. As a positive control, U937 cells were treated for 0, 12, and 24 hours with Vitamin D₃. In addition, bone marrow-derived macrophages were generated by culturing NHBM cells for two weeks in the presence of GM-CSF (10 ng/ml) and IL-3 (10 ng/ml), and these cells were treated with Vitamin D₃ for 72 hours. Total RNA was prepared from each of the four cell types, and cDNAs were synthesized by reverse transcription. The cDNAs were then analyzed by quantitative real-time RT-PCR using fluorescent probes against either hCAP18 or 18S. PCR was performed in triplicate for each sample (FIG. 10). Standard curves were created using samples with known amounts of hCAP18 or 18S cDNA, and these standard curves were used to determine the amount of hCAP18 and 18S cDNA in each sample. The X-axis of these graphs represents the amount of CAMP cDNA in each sample divided by the amount of 18S cDNA in each sample (CAMP(ng)/18S(ng)), +/−SD. The fold-change for each sample set is indicated by the number within the bar. As demonstrated previously, a strong induction of CAMP was observed for U937 treated with 1,25(OH)₂D₃ (FIG. 10A). Similarly strong induction of CAMP was observed in two normal human bone marrow cell samples and in BM Mφ (FIG. 10A). The AML cells had a high baseline level of CAMP expression, which was induced further in a dose-responsive manner by 6- and 11-fold (FIG. 10B). These data demonstrate that 1,25(OH)₂D₃ can markedly enhance the expression level of CAMP mRNA in normal and diseased human BM cells and that the induction is not a cell line phenomenon. These results also indicate that Vitamin D₃ and its analogs may be used to induce transcription of the CAMP gene in vivo.

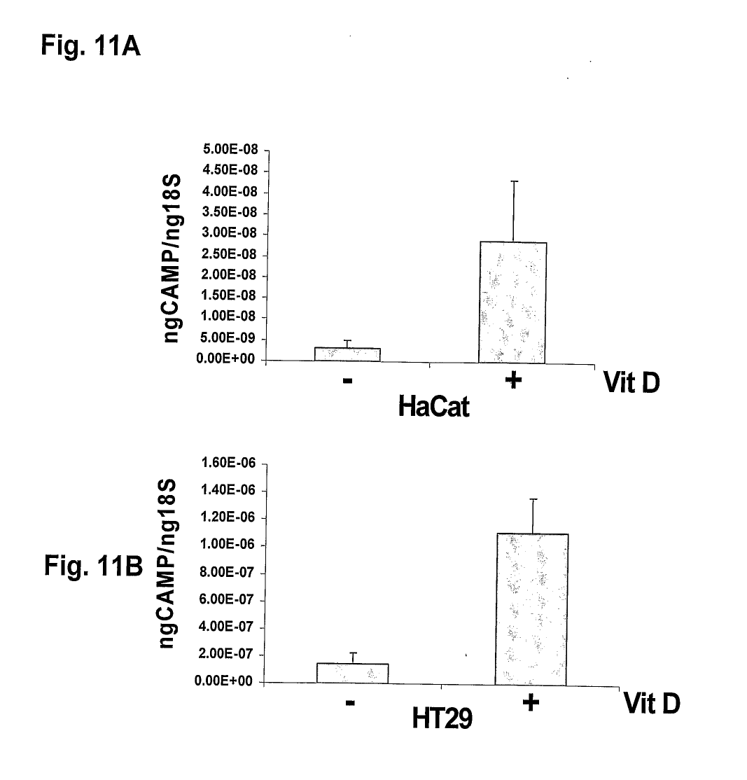

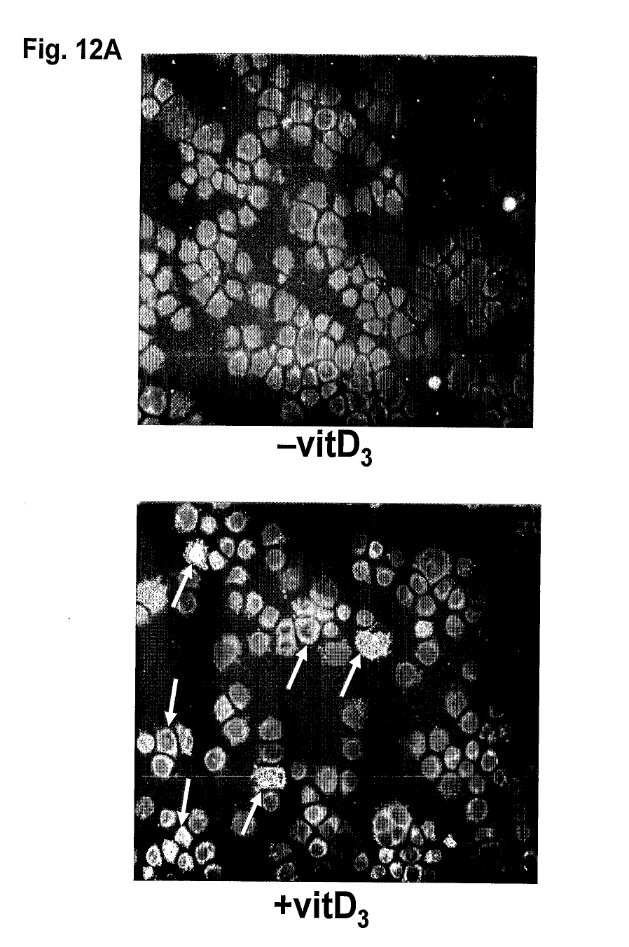

The induction of CAMP by 1,25(OH)₂D₃ was not limited to myeloid cells. Induction of CAMP mRNA was observed in the keratinocyte cell line, HaCat, and the colon cancer cell line, HT-29, by QRT-PCR (FIG. 11). However, the induction was not as robust as that observed in the myeloid cells.

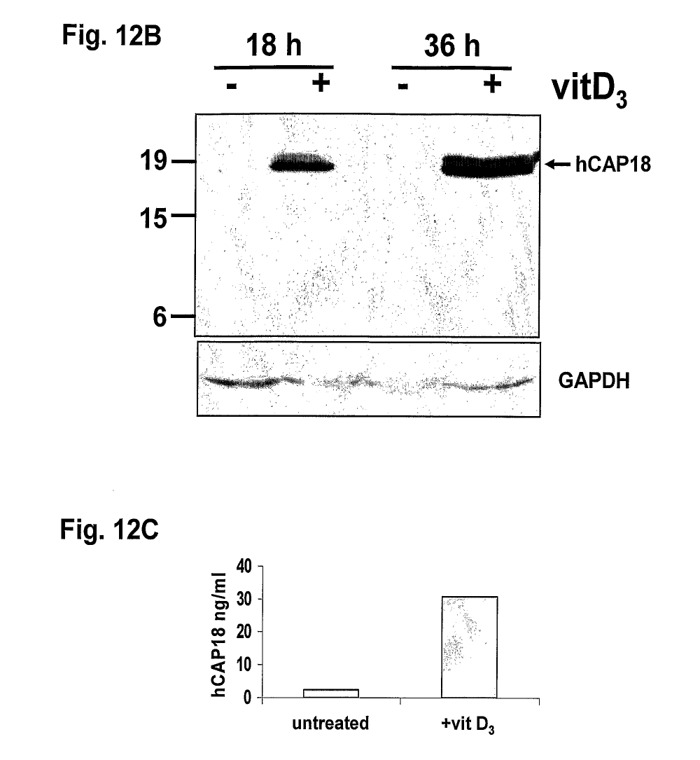

To determine if the induction of CAMP mRNA expression resulted in an increase of CAMP (hCAP18) protein expression, Western blot and immunofluorescent microscopy analyses were performed on U937 cells treated with 1,25(OH)₂D₃ (FIGS. 12A and B). At both 18 h and 36 h post treatment, increased levels of hCAP18 were observed as compared with untreated cells (FIGS. 12A and B). An ELISA performed on the medium from U937 cells treated for 24 h with either ethanol or 1,25(OH)₂D₃ showed that CAMP was secreted into the medium (FIG. 12C).

### Example 8

### Identification of a Vitamin D₃ Responsive Element (VDRE) in the CAMP Gene Promoter

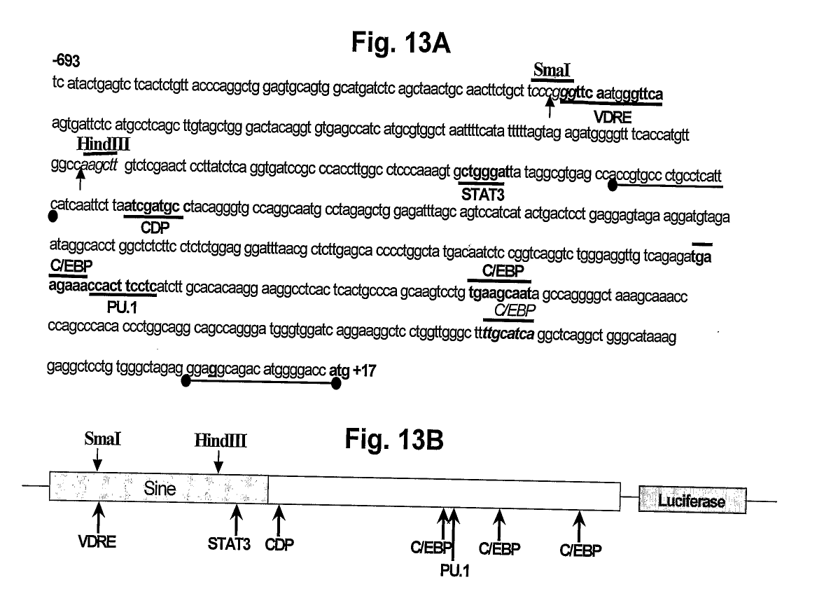

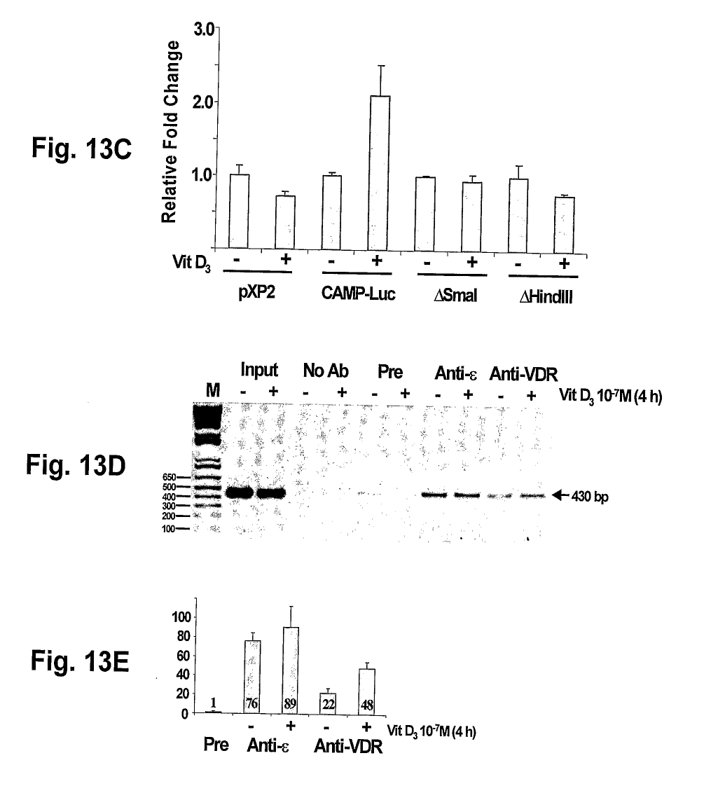

While not wishing to be bound by any theory, the existence of a VDRE in the CAMP promoter may explain the strong induction of CAMP mRNA expression by exposure to vitamin D₃. A search of the upstream region revealed a classical DR3-type VDRE (Toell, A. et al., “All natural DR3-type vitamin D response elements show a similar functionality in vitro,” Biochem J, Vol. 352, pp. 301-309 (2000)) at −615 bp from the transcriptional start site (FIGS. 13A and B) (Larrick, J. W. (1996)). In addition to this perfect consensus DR3 VDRE, consisting of two six-nucleotide repeats separated by a three-nucleotide spacer, a variety of potential binding sites for myeloid-specific transcription factors were identified, including binding sites for CCAAT/enhancer binding proteins (C/EBP), CCAAT/displacement proteins (CDP), STAT3, and PU.1 (FIGS. 13A and B). PCR was used to amplify the human CAMP promoter from nucleotides −693 to +14 (Larrick, J. W. (1996)). This fragment was subcloned into the firefly luciferase reporter plasmid pXP2 and called pXP2-CAMP-Luc (FIGS. 13A and B). Subsequently, deletion mutants pXP2-CAMP(ΔSmaI)-Luc and pXP2-CAMP(ΔHindIII)-Luc were generated by restriction enzyme digestion using the SmaI and HindIII sites, respectively (FIGS. 13A and B).

The CAMP-promoter constructs were transfected into U937 cells that were subsequently treated with vehicle or 1,25(OH)₂D₃. After 18 h treatment, cell lysates were prepared and dual luciferase assays were performed. In the absence of 1,25(OH)₂D₃, luciferase activity for all reporter constructs including the empty parental vector was similarly low (FIG. 13C). This is consistent with the very low levels of endogenous CAMP mRNA expression in untreated U937. Upon treatment, the full-length promoter construct pXP2-CAMP-Luc was consistently activated 2-2.5-fold (FIG. 13C). The deletion mutants PXP2-CAMP(ΔSmaI)-Luc and pXP2-CAMP(ΔHindIII)-Luc were not activated. Interestingly, pXP2-CAMP(ΔSmaI)-Luc still possesses the VDRE; however, the SmaI site used for the generation of the construct is immediately adjacent to the VDRE (FIGS. 13A and B) suggesting that a single or several nucleotides located 5′ to the VDRE is required for the response. These data demonstrate that this VDRE is required for activation of the CAMP promoter by vitamin D₃.

### Example 9

### VDR Binds to the CAMP Promoter in Cells

To determine if VDR complexes were actually binding to the CAMP promoter, ChIP assays were performed on chromatin prepared from U937 cells treated either with vehicle or 1,25(OH)₂D₃ for 4 h (FIGS. 13D and E). Because the VDRE is located in a repetitive DNA element or short interspersed nuclear element (SINE), it was difficult to design primers for PCR that specifically amplified that region of the CAMP promoter (FIGS. 13A and B, shaded boxes). Therefore, primers were designed corresponding to the non-repetitive region near the transcriptional start site that specifically amplifies the CAMP promoter (FIGS. 13A and B). Approximately 1×10⁷ U937 cells were incubated for four hours in the presence (“+”) or absence (“−”) of 1×10⁻⁷ M Vitamin D₃. Protein/DNA complexes, including the VDR/VDRE complex and the C/EBPε/C/EBP complex, were cross-linked in 1% formaldehyde for 10 minutes. The cross-linking reaction was terminated by the addition of glycine to 0.125 M final concentration. Cells were washed in ice-cold PBS containing PMSF (10 μg/ml), resuspended in 1 ml of SDS-lysis buffer containing protease inhibitors, and incubated on ice for 10 minutes. The lysates were sonicated three times for 10 seconds at 30% output to shear the DNA. The sonicated lysate was pelleted at 13K rpm for 10 minutes at 4° C. 200 μl of supernatant was mixed with 1.8 ml of dilution buffer and precleared with protein A-agarose for one hour on ice. Anti-C/EBPε antibody, anti-VDR antibody, or preimmune serum was added, and the sample was incubated overnight at 4° C. ssDNA/protein A-agarose slurry was then added, and this mixture was incubated overnight at 4° C. The agarose/antibody/protein/DNA complex was pelleted and washed in low salt (1×), high salt (1×), LiCl (1×), and TE (1×). The complex was removed from the protein A-agarose in elution buffer (2×500 μl), and cross-linking was reversed in 100 mM NaCl at 65° C. for four hours. The complex was treated with proteinase K, and subjected to phenol/chloroform extraction and ethanol precipitation to isolate DNA.

C/EBPε activates CAMP gene expression (Gombart, A. F. et al., “Neutrophil-specific granule deficiency: homozygous recessive inheritance of a frameshift mutation in the gene encoding transcription factor CCAAT/enhancer binding protein—epsilon,” Blood, Vol. 97, pp. 2561-2567 (2001)) and was included as a positive control. For negative controls chromatin was immunoprecipitated either with protein A-sepharose (No Ab) or preimmune serum (Pre). The samples were amplified by conventional PCR and visualized by ethidium bromide staining (FIG. 13D) or QRT-PCR (FIG. 13E). Extremely low background levels were detected in the negative controls (FIGS. 13D and E, No Ab or Pre). A significant level of the promoter was immunoprecipitated by anti-VDR Ab (22-fold above background) without 1,25(OH)₂D₃ treatment, and this increased more than 2-fold (48-fold above background) with treatment (FIG. 13E). The binding of C/EBPε to the promoter was similar under both conditions (76- and 89-fold) demonstrating that vitamin D₃ treatment is not increasing the amount of C/EBPε binding to the promoter (FIGS. 13D and E). These results indicated that VDR is binding to the CAMP promoter in both a ligand-dependent and -independent manner consistent with current models of steroid-hormone gene regulation.

### Example 10

### Induction of CAMP by Vitamin D₃ is Not Evolutionarily Conserved

To elucidate further the role of the VDR in regulating CAMP gene expression, the expression of the murine CAMP/CRAMP gene was examined in RNA from untreated bone marrow cells from a VDR-deficient mouse and its wild type littermate (FIG. 14A, left panel). Bone marrow RNAs from C/EBPε-deficient and wild type mice were included as controls (FIG. 14A, left panel). As expected, the C/EBPε-deficient bone marrow lacked expression of CRAMP (Verbeek, W. et al., “Myeloid transcription factor C/EBPepsilon is involved in the positive regulation of lactoferrin gene expression in neutrophils,” Blood, Vol. 94, pp. 3141-3150 (1999)). In contrast, CRAMP was expressed in the VDR-deficient cells at a level comparable to the wild type littermate. Furthermore, intraperitoneal treatment of BNX mice with 1,25(OH)₂D₃ or vitamin D₃ compound I over a 6-week period did not significantly alter CRAMP expression in bone marrow when compared with a vehicle-treated mouse (FIG. 14A, middle panel). In addition, we did not observe induction of CRAMP in murine cell lines 32Dcl3 (FIG. 14A, right panel), NIH3T3 and Wehi3B (data not shown). Finally, CRAMP induction was not observed in C/EBPε-deficient or wild type bone marrow cells cultured in vitro with 1,25(OH)₂D₃ (FIG. 14B) or in BM Mφ from VDR-deficient or wild type mice (FIG. 14C). Indeed, an approximately 2-fold decrease was observed by 24 h post-treatment (FIGS. 14B and C) and 5-fold by 48 h (FIG. 14C).

The genomes from human, chimpanzee, rat, dog and mouse were compared to determine the conservation of the promoter region for each CAMP gene (FIG. 14D). While significant homology was observed, a gap was identified at −409 bp upstream from the start site of transcription in the human promoter. This was a due to a SINE conserved only in the human and chimpanzee genomes and absent in the others (FIG. 14D). The VDRE is located in this SINE. Thus, the mouse gene lacks a VDRE. This is consistent with the observed absence of CRAMP induction by vitamin D₃.

### Example 11

### Vitamin D₃ Analog-Mediated Induction of hCAP18 mRNA Expression in AML Cell Lines

Because Vitamin D₃ can cause hypercalcemia, a number of analogs have been developed that are significantly less calcemic (Peleg 2003). Three Vitamin D₃ analogs (KH1060, EB 1089, and 1) were tested to determine whether they could induce hCAP18 expression as effectively as Vitamin D₃. U937 cells were treated with Vitamin D₃ (labeled “Vit D3” in FIG. 6) or one of the Vitamin D₃ analogs at a dosage of 1×10⁻⁷ M for various time periods. Total RNA was prepared and subjected to Northern analysis as described in Example 1 (above). A probe specific for β-actin was used as a control. mRNA expression was measured at 12 hours and 24 hours. Induction of hCAP18 mRNA expression was observed for each of the three Vitamin D₃ analogs tested (FIG. 6, upper panel). The levels of induction were similar to those seen with Vitamin D₃, suggesting that Vitamin D₃ analogs are just as effective as Vitamin D₃ at inducing hCAP18 mRNA expression.

While a number of exemplary aspects and embodiments have been discussed above, those of skill in the art will recognize certain modifications, permutations, additions, equivalents and changes thereof. It is therefore intended that the following appended claims and claims hereafter introduced are interpreted to include all such modifications, permutations, additions, equivalents and changes as are within their true spirit and scope. All references cited herein are incorporated by reference as if fully set forth herein.

Abbreviations used herein: VDR, Vitamin D receptor; VDRE, Vitamin D response element; hCAP18, human cationic antibacterial protein of 18 kDa; CAMP, cathelicidin antimicrobial peptide; LPS, lipopolysaccharide; Vitamin D3, 1α,25(OH)₂D₃; TPA, 12-0-tetradecanoylphorbol-13-acetate; ChIP, chromatin immunoprecipitation.

What is claimed is:

1. A method of treating a condition or promoting a process in a subject, comprising:
providing a composition comprising Vitamin D₃, one or more Vitamin D₃ analogs, or a combination of Vitamin D₃ and one or more Vitamin D₃ analogs; and
administering said composition to said subject to induce cathelicidin production in said subject to a level sufficient to treat said condition or promote said process.

2. The method of claim 1, wherein said cathelicidin is hCAP18.

3. The method of claim 1, wherein said condition is selected from the group consisting of microbial infections, skin infections, infections of the colon, sepsis and combinations thereof, and said process is selected from the group consisting of wound healing, angiogenesis, chemoattraction and combinations thereof.

4. The method of claim 3, wherein said subject is a mammal.

5. The method of claim 3, wherein said subject is a human.

6. The method of claim 1, wherein the route of said administration is topical, transdermal, or parenteral.

7. The method of claim 1, wherein said one or more Vitamin D₃ analogs are selected from the group consisting of calcipotriol (MC903), maxacalcitol (OCT), paricalcitol, tacalcitol, doxercalciferol, alfacalcidol, seocalcitol (EB1089), SM-10193, EB1072, EB1129, EB1133, EB1155, EB1270, MC1288, EB1213, CB1093, CB966, VD2656, VD2668, VD2708, VD2716, VD2728, VD2736, GS1500, GS1558, KH1060, ZK161422, and Vitamin D₃ analog I.

8. The method of claim 1, wherein said one or more Vitamin D₃ analogs are selected from the group consisting of lexacalcitol (KH1060), seocalcitol (EB1089), and Vitamin D₃ analog I.

9. The method of claim 5, wherein said induction of said cathelicidin occurs at the site of the skin infection, the infection of the colon, the sepsis, the microbial infection, or the wound, and the skin infection, the infection of the colon, the sepsis, the microbial infection, or the wound occurs in the neutrophils, plasma, epithelial cells, or oral cavity of the human.

10. The method of claim 3, wherein said induction of said cathelicidin occurs at a site other than the site of the skin infection, the infection of the colon, the sepsis, the microbial infection, or the wound.

11. The method of claim 3, wherein said induction results in the cathelicidin reaching the site of the skin infection, the infection of the colon, the sepsis, the microbial infection, or the wound by traveling through the circulatory system.

12. The method of claim 1, wherein said composition includes a pharmaceutically acceptable carrier.

13. A method of inducing endogenous cellular cathelicidin production, comprising:
providing a composition comprising Vitamin D₃, one or more Vitamin D₃ analogs, or a combination of Vitamin D₃ and one or more Vitamin D₃;
administering said composition in an amount sufficient to induce endogenous cellular production of cathelicidin.

14. The method of claim 13, wherein said cathelicidin is hCAP18.

15. The method of claim 13, wherein said one or more Vitamin D₃ analogs are selected from the group consisting of calcipotriol (MC903), maxacalcitol (OCT), paricalcitol, tacalcitol, doxercalciferol, alfacalcidol, seocalcitol (EB1089), SM-10193, EB1072, EB1129, EB1133, EB1155, EB1270, MC1288, EB1213, CB1093, CB966, VD2656, VD2668, VD2708, VD2716, VD2728, VD2736, GS1500, GS1558, KH1060, ZK161422, and Vitamin D₃ analog I.

16. The method of claim 13, wherein said one or more Vitamin D₃ analogs are selected from the group consisting of lexacalcitol (KH1060), seocalcitol (EB1089), and Vitamin D₃ analog I.

17. A method of treating microbial infections, skin infections, infections of the colon, sepsis, or combinations thereof in a subject, comprising administering Vitamin D₃, one or more Vitamin D₃ analogs, or a combination of Vitamin D₃ and one or more Vitamin D₃ analogs in an amount sufficient to treat said microbial infections, skin infections, infections of the colon, sepsis, or combinations thereof.

18. A method of promoting wound healing, angiogenesis, chemoattraction, or combinations thereof in a subject, comprising administering Vitamin D₃, one or more Vitamin D₃ analogs, or a combination of Vitamin D₃ and one or more Vitamin D₃ analogs in an amount sufficient to promote said wound healing, angiogenesis, chemoattraction, or combinations thereof.

19. A method of treating a condition or promoting a process in a subject, comprising:
providing a composition comprising Vitamin D₃, one or more Vitamin D₃ analogs, or a combination of Vitamin D₃ and one or more Vitamin D₃ analogs; and
administering said composition to said subject to induce defensin production in said subject to a level sufficient to treat said condition or promote said process.

20. The method of claim 19, wherein said defensin is a defensin β2 gene product.

21. The method of claim 19, wherein said condition is selected from the group consisting of microbial infections, skin infections, infections of the colon, sepsis and combinations thereof, and said process is selected from the group consisting of wound healing, angiogenesis, chemoattraction and combinations thereof.

22. The method of claim 21, wherein said subject is a mammal.

23. The method of claim 21, wherein said subject is a human.

24. The method of claim 19, wherein the route of said administration is topical, transdermal, or parenteral.

25. The method of claim 19, wherein said one or more Vitamin D₃ analogs are selected from the group consisting of calcipotriol (MC903), maxacalcitol (OCT), paricalcitol, tacalcitol, doxercalciferol, alfacalcidol, seocalcitol (EB1089), SM-10193, EB1072, EB1129, EB1133, EB1155, EB1270, MC1288, EB1213, CB1093, CB966, VD2656, VD2668, VD2708, VD2716, VD2728, VD2736, GS1500, GS1558, KH1060, ZK161422, and Vitamin D₃ analog I.

26. The method of claim 19, wherein said one or more Vitamin D₃ analogs are selected from the group consisting of lexacalcitol (KH1060), seocalcitol (EB1089), and Vitamin D₃ analog I.

27. The method of claim 23, wherein said induction of said cathelicidin occurs at the site of the skin infection, the infection of the colon, the sepsis, the microbial infection, or the wound, and the skin infection, the infection of the colon, the sepsis, the microbial infection, or the wound occurs in the neutrophils, plasma, epithelial cells, or oral cavity of the human.

28. The method of claim 21, wherein said induction of said cathelicidin occurs at a site other than the site of the skin infection, the infection of the colon, the sepsis, the microbial infection, or the wound.

29. The method of claim 21, wherein said induction results in the cathelicidin reaching the site of the skin infection, the infection of the colon, the sepsis, the microbial infection, or the wound by traveling through the circulatory system.

30. The method of claim 19, wherein said composition includes a pharmaceutically acceptable carrier.


SEQUENCE LISTING (10 sequences)

SEQ ID NO: 1 - DNA, 714 bp, Homo sapiens
tcatactgagtctcactctgttacccaggctggagtgcagtggcatgatctcagctaactgcaacttctgcttcccgggttcaatgggttcaagtgattctcatgcctcagcttgtagctgggactacaggtgtgagccatcatgcgtggctaattttcatatttttagtagagatggggtttcaccatgttggccaagcttgtctcgaactccttatctcaggtgatccgcccaccttggcctcccaaagtgctgggattataggcgtgagccaccgtgccctgcctcattcatcaattcttaatcgatgcctacagggtgccaggcaatgcctagagctggagatttagcagtccatcatactgactcctgaggagtagaaggatgtagaataggcacctggctctcttcctctctggagggatttaacgctcttgagcacccctggctatgacaatctccggtcaggtctgggaggttgtcagagatgaagaaaccacttcctcatcttgcacacaaggaaggcctcactcactgcccagcaagtcctgtgaagcaatagccaggggctaaagcaaaccccagcccacaccctggcaggcagccagggatgggtggatcaggaaggctcctggttgggcttttgcatcaggctcaggctgggcataaaggaggctcctgtgggctagagggaggcagacatggggaccatg

SEQ ID NO: 2 - DNA, 33 bp, Unknown
  Probe/primer
ccgacgcgtcatactgagtctcactctgttacc

SEQ ID NO: 3 - DNA, 27 bp, Unknown
  Probe/primer
ccgctcgagggtccccatgtctgcctc

SEQ ID NO: 4 - DNA, 20 bp, Unknown
  Probe/primer
accccaggcccacgatggat

SEQ ID NO: 5 - DNA, 20 bp, Unknown
  Probe/primer
gctaacctctaccgcctcct

SEQ ID NO: 6 - DNA, 20 bp, Unknown
  Probe/primer
ggtcactgtccccatacacc

SEQ ID NO: 7 - DNA, 19 bp, Unknown
  Probe/primer
gcagttccagagggacgtc

SEQ ID NO: 8 - DNA, 20 bp, Unknown
  Probe/primer
gttccttgaaggcacattgc

SEQ ID NO: 9 - DNA, 19 bp, Unknown
  Probe/primer
accgtgccctgcctcattc

SEQ ID NO: 10 - DNA, 19 bp, Unknown
  Probe/primer
tggtccccatgtctgcctc